(19) United States
(12) Patent Application Publication
(10) Pub. No.: US 2019/0147976 A1
(43) Pub. Date: May 16, 2019

(54) CHECKPOINT FAILURE AND METHODS THEREFOR

(71) Applicant: NantOmics, LLC, Culver City, CA (US)

(72) Inventor: Charles Joseph Vaske, Culver City, CA (US)

(73) Assignee: NantOmics, LLC, Culver City, CA (US)

(21) Appl. No.: 16/098,611

(22) PCT Filed: May 5, 2017
(86) PCT No.: PCT/US17/31418

§ 371 Date: Nov. 2, 2018

Related U.S. Application Data
(60) Provisional application No. 62/332,047, filed on May 5, 2016.

(51) Int. Cl. G16B 30/00; G16B 20/00; G16H 10/60; C12Q 1/6886

(52) U.S. Cl. CPC G16B 30/00; G16B 20/00; G16H 10/60; C12Q 1/6886; C12Q 2600/136; C12Q 2600/154; C12Q 2600/112; C12Q 2600/178; C12Q 2600/158

(57) ABSTRACT

Systems and methods for more accurate prediction of the treatment outcome for immune therapy using checkpoint inhibitors are presented in which omics data of a patient tumor sample are used. In one aspect, a pathway signature is identified as being associated with immune suppression and as being responsive to treatment with immune checkpoint inhibitors.

This application claims priority to U.S. provisional application Ser. No. 62/332,047, filed May 5, 2016. U.S. application No. 62/332,047 is incorporated herein in its entirety.

### FIELD OF THE INVENTION

The field of the invention is computational analysis of various omics data to allow for treatment stratification for immune therapy, and especially pathway-based analysis to identify likely responders to checkpoint inhibitor treatment.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Immune therapy with genetically modified viruses has become increasingly effective and attractive route for treatment of various cancers. However, several challenges remain to be resolved. For example, the choice of suitable antigens to be expressed is non-trivial (see e.g., Nat Biotechnol. 2012; 30(7):658-70; and Nat Biotechnol. 2017; 35(2): 79). Moreover, even frequently or highly expressed epitopes will not guarantee a tumor-protective immune reaction in all patients. In addition, even where several neoepitopes are known and used as an immunotherapeutic composition, inhibitory factors in the tumor microenvironment may nevertheless prevent a therapeutically effective response. For example, a sufficient immune response may be blunted or even prevented by Tregs (i.e., regulatory T cells) and/or MDSCs (myeloid derived suppressor cells). In addition, lack of stimulatory factors and tumor based interference with immune checkpoints, and especially PD-1 and CTLA-4, may still further prevent a therapeutic response to immune therapy.

Therapeutic compositions are known to block or silence immune checkpoints (e.g., Pembrolizumab or Nivolumab for the PD-1 system, or Ipilimumab for the CTLA-4 system). However, administration is not consistently effective to promote a durable and therapeutically useful response. Likewise, cyclophosphamide may be used to suppress Tregs, however tends to mobilize MDSCs. Thus, a clear path to intervention in patients with low immune response to immune therapy is not apparent. More recently, a predictive model was proposed that used levels of tumor MHC class I expression as a positively correlated marker with overall tumor immunogenicity (see J Immunother 2013, Vol. 36, No 9, p 477-489). The authors also noted a pattern where certain immune activating genes were up-regulated in strongly immunogenic tumors of some of the models, but advised that additional biomarkers should be found to help predict immunotherapy response. In another approach (Cancer Immunol Res; 4(5) May 2016, OF1-7), post-treatment in depth sequence and distribution analysis of tumor reactive T cell receptors was used as a proxy indicator for reactive T-cell tumor infiltration. Unfortunately, such analysis fails to provide predictive insight with respect to likely treatment success for immune therapy.

In still further known approaches, change in expression level of selected genes was used as a signature predictive of increased likelihood of being responsive to immunotherapy as described in WO 2016/109546. Similarly, US 2016/0312295 and US 2016/0312297 teach gene signature biomarkers that are useful for identifying cancer patients who are most likely to benefit from treatment with a PD-1 antagonist. While such signatures tend to be at least somewhat informative, they are generally ‘static’ and typically fail to reflect pathway activity that could be indicative of sensitivity and/or susceptibility to treatment with one or more checkpoint inhibitors.

Thus, even though various systems and methods of immune therapy and checkpoint inhibition are known in the art, all or almost all of them suffer from several drawbacks. Therefore, there is still a need to provide improved compositions and methods to identify patients that are responsive to immune therapy and treatment with checkpoint inhibitors.

### SUMMARY OF THE INVENTION

The inventive subject matter is directed to computational analysis of omics data to predict likely treatment success to immune therapy using checkpoint inhibitors. In one particularly preferred aspect, computational pathway analysis is performed on omics data obtained from a tumor sample (e.g., breast cancer tumor sample containing tumor infiltrating lymphocytes), wherein the pathway analysis uses a cluster of features and pathways that are associated with specific subsets of immune related genes. In still further preferred aspects, the features and pathways are associated with an up-regulated FOXM1 signaling pathway, with the presence and/or inhibition of tumor infiltrating lymphocytes, with a low (as compared to healthy tissue) Th1/Th2 ratio, and with a basal-like character.

In one aspect of the inventive subject matter, the inventors contemplate a method of predicting a likely therapeutic outcome for immune therapy of a cancer with a checkpoint inhibitor (e.g., CTLA-4 or a PD-1 inhibitor). Preferred methods comprise a step of obtaining omics data from a tumor of the patient, wherein the omics data comprise at least one of whole genome sequencing data and RNA sequencing data, and a further step of using pathway analysis to identify from the omics data a plurality of highly expressed genes in a plurality of immune related pathways having a plurality of respective pathway elements. In another step, the highly expressed genes are associated with a likely response of the cancer to treatment with the checkpoint inhibitor when the highly expressed genes are indicative of a Th2/humoral response and a low Th1/Th2 ratio, and in a still further step, a patient record is updated or generated record with an indication of the likely response of the cancer to treatment with the checkpoint inhibitor when the highly expressed genes are indicative of a Th2/humoral response and a low Th1/Th2 ratio.

Preferred immune related pathways include an immune cell function pathway, a pro-inflammatory signaling pathway, and an immune suppression pathway, and/or the pathway element controls activity of Th1 differentiation, Th2 differentiation, B cell differentiation, macrophage differentiation, T cell activation, and/or an immunoproteasome. For example, while some contemplated pathway elements will control activity of NFkB, and/or IFNalpha responsive gen, other pathway elements include cytokines, and especially IL12 beta, IFNgamma, IL4, IL5, and IL10. Further contemplated pathway elements include one or more chemokines, including CCL17, CCL11, and CCL26.

Therefore, and among other suitable pathway elements, especially contemplated elements are selected form the group consisting of IL12B, IFNG, PSMA3, THY1, CCL17, PRKCQ, NFATC3, NFATC2, CCL11, CCL26, IFNAR2, SQSTM1, IRAK4, NFKBIA, IL6ST, MAP3K1, IRF1, IRF9, PTGS2, IL4, IL5, IGHG3, IL4R, IL13RA2, PIGR, IL13RA1, STAT6, FCER2, IGHG1, IL10, STAT5A, PRKCE, CSF1R, ARG1, LTA, SELP, FKBP3, LCP2, and DOK2. Where the pathway element is a complex, especially contemplated complexes are selected form the group consisting of IFN-gamma/IRF1, STAT6 (dimer)/PARP14, IL4/IL4R/JAK1, IL4R/JAK1, STAT6 (dimer)/ETS1, PI3K/BCAP/CD19, IL4/IL4R/JAK1/IL2Rgamma/JAK3/DOK2, IL4/IL4R/JAK1/IL2Rgamma/JAK3/SHIP, IL4/IL4R/JAK1/IL13RA1/JAK2, IL4/IL4R/JAK1/IL2Rgamma/JAK3/SHC/SHIP, IL4/IL4R/JAK1/IL2Rgamma/JAK3/FES/IRS2, IL4/IL4R/JAK1/IL2Rgamma/JAK3, IL4/IL4R/JAK1/IL2Rgamma/JAK3/SHC/SHIP/GRB2, IL4/IL4R/JAK1/IL2Rgamma/JAK3/IRS1, IL4/IL4R/JAK1/IL2Rgamma/JAK3/FES, IL4/IL4R/JAK1/IL2Rgamma/JAK3/SHP1.

In further contemplated aspects, the omics data may further comprise siRNA data, DNA methylation status data, transcription level data, and/or proteomics data. Most preferably, the pathway analysis comprises PARADIGM analysis, and/or the omics data are normalized against the same patient (before or after treatment). Typically, the cancer is a breast cancer, and the highly expressed genes will further include FOXM1. However, contemplated highly expressed genes may further include non-immune genes encoding a protein involved in at least one of mitogenic signaling, stress signaling, apoptosis, calcium/calmodulin signaling, G-protein signaling, PI3K/AKT signaling, RTK signaling, Wnt signaling, and cAMP signaling, non-immune genes encoding a protein involved in at least one of cell cycle control, DNA damage response, and chromatin remodeling, and/or non-immune genes selected from the group consisting of MAPK1, MAPK14, NRP2, HIF1A, CALM1, CREB1, CSNK1A1, CSNK1G3, CCNH, FANCE, FANCA, TFIIH, ITGB3, RASA1, GNG2, PDGFRB, AKT1, and PIK3R1. In further contemplated methods, the likely therapeutic outcome is predicted prior to therapy with the checkpoint inhibitor, and/or the immune therapy may further comprise administration of at least one of a genetically modified virus and a genetically modified NK cell.

Various objects, features, aspects and advantages of the inventive subject matter will become more apparent from the following detailed description of preferred embodiments, along with the accompanying drawing.

### DETAILED DESCRIPTION

The inventors have discovered systems and methods of predicting a likely treatment outcome of cancer immune therapy by computational analysis of pathway signatures found in tumor tissue to identify the immune status of a tumor. In especially preferred aspects of the inventive subject matter, positive treatment outcome with checkpoint inhibitors is predicted in breast cancer where a tumor has attributes of an up-regulated FOXM1 signaling pathway, with presence and/or inhibition of tumor infiltrating lymphocytes, with a low (as compared to healthy tissue) Th1/Th2 ratio, and with a basal-like character.

In this context, it should be appreciated that contemplated systems and methods take advantage of differentially expressed genes (using mRNA quantity and copy number as the main contributors) in pathways versus the same genes in healthy tissue as predictor. Most typically, differentially expressed genes will be up-regulated relative to the same genes in healthy tissue, however, down-regulated genes are also contemplated (and often present in genes associated with Th1 phenotype). Moreover, it should also be recognized that pathway analysis (e.g., using PARADIGM) provides a significant advantage in such analysis identifies active pathways in subsets of patients that would otherwise be indistinguishable where genes are studied at a single level. Particularly preferred methods of pathway analysis make use of techniques from probabilistic graphical models to integrate functional genomics data onto a known pathway structure. Such analysis not only provides better discrimination of patients with respect to prognosis than any of the molecular levels studied separately, but also allows for identification of immune status of a tumor based on characteristics that are reflected in specific immune related pathway activities, and particularly with FOXM1 signaling pathway activity, activity of Th1 and Th2 related pathways, pathway activity associated with innate immunity, and pathways associated with sub-type of cancer (e.g., luminal, basal). Indeed, clustering of results from pathway analysis revealed distinct groups of differential pathway activity as is discussed in more detail below.

For example, and as discussed in more detail below, the inventors observed that all clusters that were associated with good outcome (increased survival time) were significantly enriched in genes associated with antitumor immunity at the expense of the Th2/humoral immune response, which is also consistent with a higher ratio of Th1/Th2 genes in these clusters. On the other hand, the cluster that was associated with poorer outcome (decreased survival time) was significantly enriched in Th2/humoral-related genes and had significantly lower Th1/Th2 ratios. Notably, the inventors discovered that the pathway activities in such cluster was also prognostic for treatment success with one or more checkpoint inhibitors.

Consequently, it is contemplated that prior to treatment (or after one round of cancer treatment but before a subsequent round of cancer treatment), a tumor biopsy is obtained from a patient and that omics analysis is performed on the so obtained sample. In general, it is contemplated that the omics analysis includes whole genome and/or exome sequencing, RNA sequencing and/or quantification, and/or proteomics analysis. Most typically, the omics analysis will also include obtaining information about copy number alterations, especially amplification of one or more genes. As will be readily appreciated, it is contemplated that genomic analysis can be performed by any number of analytic methods, however, especially preferred analytic methods include next generation WGS (whole genome sequencing) and exome sequencing of both a tumor and a matched normal (healthy tissue of same patient) sample. Alternatively, the matched normal sample may also be replaced in the analysis by a reference sample (typically representative of healthy tissue). Moreover, the matched normal or reference sample may be from the same tissue type as the tumor or from blood or other non-tumor tissue.

Computational analysis of the sequence data may be performed in numerous manners. In most preferred methods, however, analysis is performed in silico by location-guided synchronous alignment of tumor and normal samples as, for example, disclosed in US 2012/0059670 and US 2012/0066001 using BAM files and BAM servers. Of course, alternative file formats (e.g., SAM, GAR, FASTA, etc.) are also expressly contemplated herein. Regardless of the manner of analysis, contemplated DNA omics data will preferably include information about copy number, patient- and tumor specific mutations, and genomic rearrangements, including translocations, inversions, amplifications, fusion with other genes, extrachromosomal arrangement (e.g., double minute chromosome), etc.

Likewise, RNA sequencing and/or quantification can be performed in all manners known in the art and may use various forms of RNA. For example, preferred materials include mRNA and primary transcripts (hnRNA), and RNA sequence information may be obtained from reverse transcribed polyA⁺-RNA, which in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods also include quantitative RNA (hnRNA or mRNA) analysis and/or quantitative proteomics analysis. Most typically, RNA quantification and sequencing is performed using qPCR and/or rtPCR based methods, although other methods (e.g., solid phase hybridization-based methods) are also deemed suitable. Therefore, and viewed from another perspective, transcriptomic analysis may be suitable (alone or in combination with genomic analysis) not only for quantification of transcripts, but also to identify and quantify genes that have tumor- and patient specific mutations.

Similarly, proteomics analysis can be performed in numerous manners, and all known manners or proteomics analysis are contemplated herein. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One example of technique for conducting proteomic assays includes U.S. Pat. No. 7,473,532 to Darfler et al. titled “Liquid Tissue Preparation from Histopathologically Processed Biological Samples, Tissues, and Cells” filed on Mar. 10, 2004. Still other proteomics analyses include mass spectroscopic assays, and especially MS analyses based on selective reaction monitoring.

The so obtained omics data are then further processed to obtain pathway activity and other pathway relevant information using various systems and methods known in the art. However, particularly preferred systems and methods include those in which the pathway data are processed using probabilistic graphical models as described in WO 2011/139345 and WO 2013/062505, or other pathway models such as those described in WO 2017/033154, all incorporated by reference herein. Thus, it should be appreciated that pathway analysis for a patient may be performed from a single patient sample and matched control (once before treatment, or repeatedly, during and/or after treatment), which will significantly improve and refine analytic data as compared to single omics analysis that is compared against an external reference standard. In addition, the same analytic methods may further be refined with patient specific history data (e.g., prior omics data, current or past pharmaceutical treatment, etc.).

Once pathway activity from the omics data of the tumor sample has been calculated, differentially activated pathways and pathway elements (e.g., relative to ‘normal or patient-specific normal) in the output of the pathway analysis are then analyzed against a signature that is characteristic for an immune suppressed tumor. Most typically, such signature has the features and pathways that are associated with an up-regulated FOXM1 signaling pathway, with the presence and/or inhibition of tumor infiltrating lymphocytes, with a low (as compared to healthy tissue) Th1/Th2 ratio, and with a basal-like character.

In one exemplary aspect, and as is discussed in more detail below, the signature of an immune suppressed tumor is based on the most significant portion (e.g., top 500 features, top 200 features, top 100 features) of pathway features from patient groups clusters identified in a machine learning environment. For example, pathway analysis was performed for breast cancer patients in which one group (MicMa) had good outcome as evidenced by overall survival while another group (Chin/Naderi) had poor outcome as evidenced by overall survival. Here, pathway analysis allowed for definition of five different clusters in which the clusters were characterized as follows: PDGM1=high FOXM1, high Th1/Th2 ratio, basal/ERBB2; PDGM2=high FOXM1, low Th1/Th2 ratio, basal; PDGM3=high FOXM1, innate immune genes, macrophage dominated, luminal; PDGM4=high ERBB4, low angiopoietin signaling, luminal; and PDGM5=low FOXM1, low macrophage signature, luminal A.

Of course, it should be appreciated that numerous other groupings and clusters can be used to differentiate likely treatment outcomes. For example, suitable clusters may be based on specific tumor types, patient sub-populations, and may be larger or smaller. Moreover, it should be noted that contemplated systems and methods may also be based on or include specific neoepitopes and/or T cell receptors with specificity to one more tumor related epitopes (e.g., neoepitopes or cancer associated epitopes). In such case, expression of a specific neoepitope (especially a HLA-matched neoepitope) may be used as a proxy marker for immunogenicity. On the other hand, expression and/or quantity of a T cell receptor that binds a specific epitope may be used as a marker for immunogenicity. Similarly, it is noted that the distribution (e.g., between tumor and circulating blood) of T cell receptors specific to a neoepitope may be used as an indicator for immunogenicity. Likewise, expression of the patient's MHC-I may be ascertained and quantified to obtain a further measure of immunogenicity. In this context, it should be appreciated that this information can be readily obtained from the omics data and that omics analysis will advantageously eliminate the need for ex vivo immune staining protocols.

Regardless of the particular clustering or grouping employed, it is contemplated that the differential pathway activities of the patient are identified and compared against the signature that is indicative of an immune suppressed tumor (comprises features and pathway activities associated with an up-regulated FOXM1 signaling pathway, with the presence and/or inhibition of tumor infiltrating lymphocytes, with a low Th1/Th2 ratio, and with a basal-like character). Such comparison may include a comparison of one or more selected features that are representative of specific pathways (e.g., identification of expression level of selected genes encoding proteins that are part of a specific signaling pathway) or may include a comparison of a set of features, where a degree of similarity is identified (e.g., at least 50%, 60%, 70%, or 80% of overexpressed genes in tumor are also overexpressed in feature set of the signature. Upon determination that the patient data match or are consistent with the signature that is characteristic for immune suppression, treatment with a checkpoint may be advised (e.g., by generating or updating a patient record with an indication that checkpoint inhibition may be effective).

### Examples

Identification of breast cancer related pathways was performed using data sets from patient populations with known history. MicMa patients with breast cancer (n=101) in this study were part of a cohort of patients treated for localized breast cancer from 1995 to 1998. Samples from the UPPSALA cohort, collected at the Fresh Tissue Biobank, Department of Pathology, Uppsala University Hospital, were selected from a population-based cohort of 854 women diagnosed between 1986 and 2004 with one of three types of primary breast cancer lesions: (a) pure DCIS, (b) pure invasive breast cancer 15 mm or less in diameter, or (c) mixed lesions (invasive carcinoma with an in situ component). The Mammographic Density and Genetics cohort, including 120 healthy women with no malignant disease but some visible density on mammograms, referred to here as healthy women, was included in this study. Two breast biopsies and three blood samples were collected from each woman. The Chin validation set consisted of 113 tumor samples with both expression (GEO accession no. GSE6757) and CGH data (MIAMEExpress accession E-Ucon-1). The UNC validation dataset consisted of 78 tumor samples with both expression (44 K; Agilent Technologies) and SNP-CGH (109 K; Illumina).

Data preprocessing and PARADIGM parameters were as follows: Copy number was segmented using circular binary segmentation (CBS) and then mapped to gene-level measurements by taking the median of all segments that span a RefSeq gene's coordinates in hg18. For mRNA expression, measurements were first probe-normalized by subtracting the median expression value for each probe. The manufacturer's genomic location for each probe was converted from hg17 to hg18 using University of California, Santa Cruz liftOver tool. Per-gene measurements were then obtained by taking the median value of all probes overlapping a RefSeq gene. Methylation probes were matched to genes using the manufacturer's description. PARADIGM was run as it previously described (Bioinformatics 26:i237ei245), by quantile-transforming each dataset separately, but data were discretized into bins of equal size rather than at the 5% and 95% quantiles. Pathway files were from the Pathway Interaction Database (Nucleic Acids Res 37: D674eD679) as previously parsed.

HOPACH unsupervised clustering: Clusters were derived using the HOPACH R implementation version 2.10 (J Stat Planning Inference 117:275e303) running on R version 2.12. The correlation distance metric was used with all data types, except for PARADIGM IPLs, which used cosangle because of the nonnormal distribution and prevalence of zero values. For any cluster of samples that contained fewer than five samples, each sample was mapped to the same cluster as the most similar sample in a larger cluster. PARADIGM clusters in the MicMa dataset were mapped to other data types by determining each cluster's mediod (using the median function) in the MicMa dataset and then assigning each sample in another dataset to whichever cluster mediod was closest by cosangle distance. The copy number was clustered on gene-level values rather than by probe. The values that went into the clustering are from the CBS segmentation of each sample. A single value was then generated for each gene by taking the median of all segments that overlap the gene. The samples were then clustered using these gene-level copy number estimates with an uncentered correlation metric in HOPACH. For display, the genes and samples were median-centered.

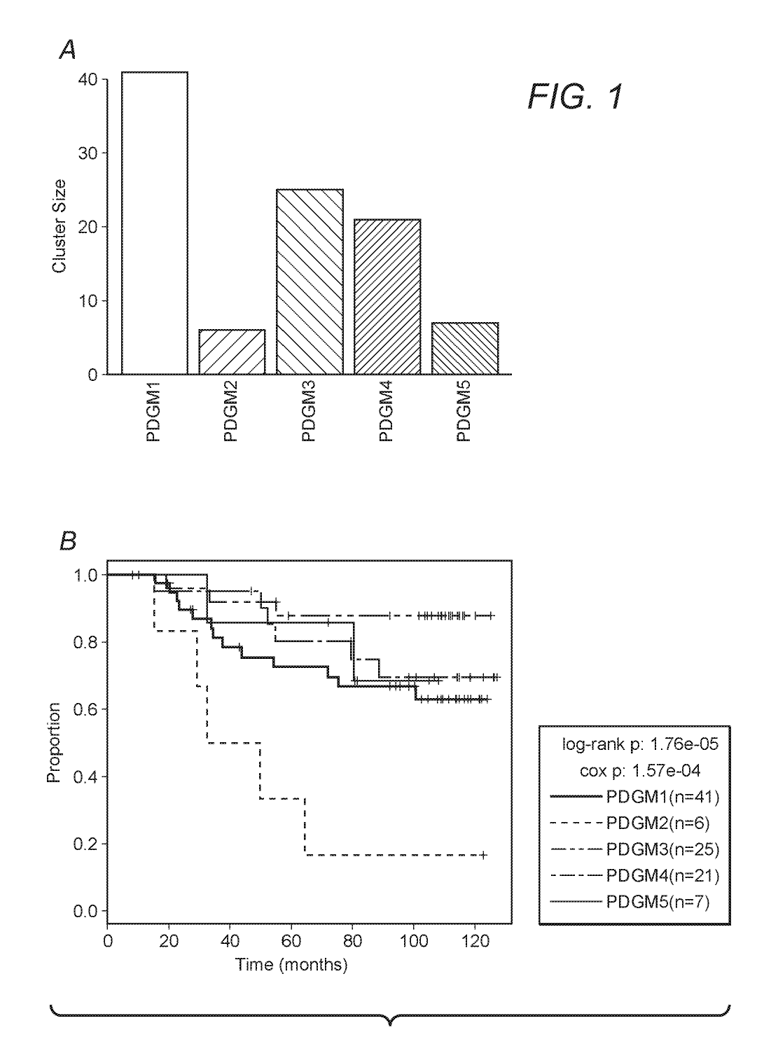

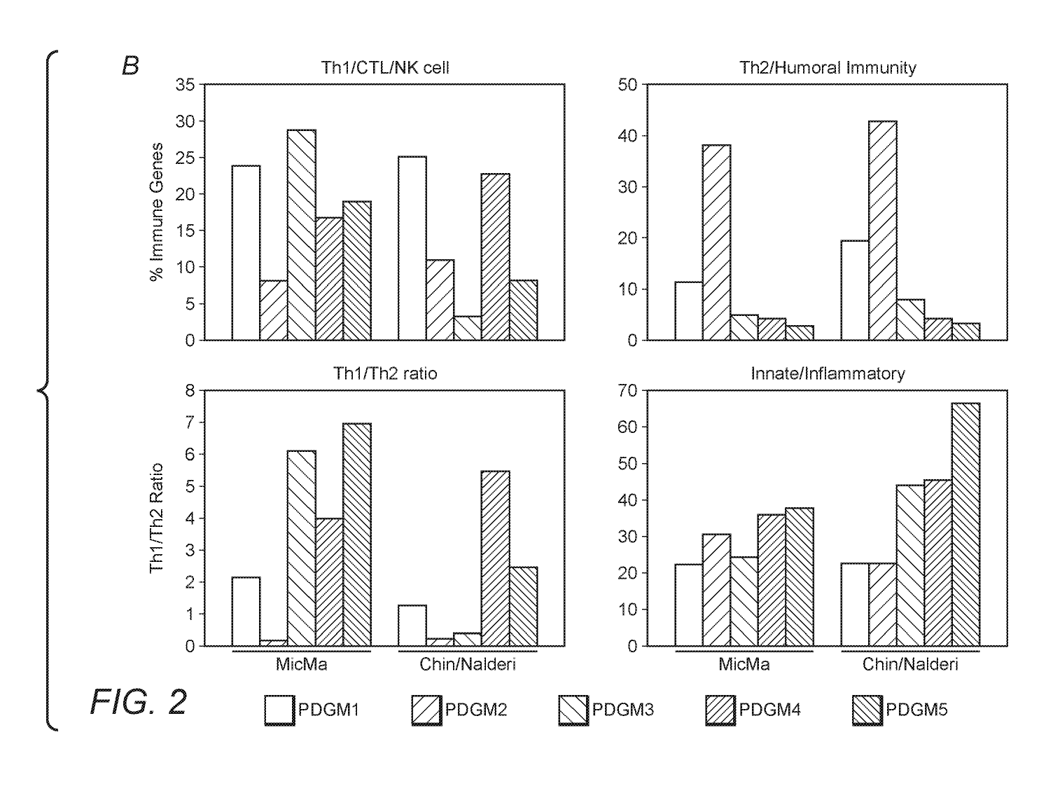

Notably, unsupervised clustering in the pathway analysis lead to a sub-typing into distinct clusters with differential survivals, and the inventors unexpectedly discovered that the genes that strongly associated with each cluster defining the subtypes were largely immune-based. Notably, genes associated with good outcome as evidenced by overall survival were found to coincide with Th1 cells and Th1 signaling, cytotoxic T cells, and natural killer cells as can be seen from FIG. 1. Moreover, genes associated with poor outcome were found to coincide with immune suppression, Th2 cells, Th2 signaling, and humoral immunity. As can be seen from panel A of FIG. 1, five distinct clusters with different sizes were identified. These clusters were defined by distinct characteristics: PDGM1 had high FOXM1, high Th1/Th2 ratio, basal/ERBB2 character; PDGM2 had high FOXM1, low Th1/Th2 ratio, and basal character; PDGM3 had high FOXM1, innate immune genes, macrophage dominated and luminal character; PDGM4 had high ERBB4, low angiopoietin signaling, and luminal character; and PDGM5 had low FOXM1, low macrophage signature, and luminal A character. Panel B of FIG. 1, illustrates the corresponding Kaplan-Meier curves. As is readily evident, best survival outcome was associated with an immunogenic and Th1-biased character (PARADIGMS), while the worst survival outcome was associated with a non-immunogenic and Th2-biased character. Notably, PARADIGM2 exhibited a pathway activity signature that reflected an immune suppressed tumor. Consequently, where omics data and corresponding pathway activities are consistent with PARADIGM2 cluster, the inventors contemplate that tumors treated with checkpoint inhibitors will be responsive to such treatment and become more immunogenic.

The most significantly differentially expressed pathways and genes that comprise the PARADIGM2 cluster are summarized in the tables below. More specifically, the tables below list exemplary immune related features within the top 500 features in the cluster that was associated with high FOXM1, low Th1/Th2 ratio, and basal character, for both good and poor outcome groups. Table 1 lists pathway entities (individual proteins or complexes) that are located in immune related pathways and that are differentially regulated relative to healthy tissue. These entities were from a subgroup of negative outcome patients.

TABLE 1

| Chin Immune-related | Function | Rank |
|---|---|---|
| PathwayEntity | Anti-tumor Immunity (NK cell, CTL, M1 macrophage | 39 |
| | function) | |
| 51_T-helper 1 cell differentiation | anti-tumor immunity | 125 |
| 9_IL12B | important for Th1 differentiation | 138 |
| 10_IL12B | important for Th1 differentiation | 170 |
| 86_IL12B | important for Th1 differentiation | 352 |
| | synergizes strongly with IL12 to trigger IFNg production of naive | 388 |
| 86_IL27RA | CD4 T cells | |
| 110_T-helper 1 cell lineage commitment | anti-tumor immunity | 392 |
| 17_STAT1 | anti-tumor immunity | 431 |
| 86_IL27RA/JAK1 | synergizes strongly with IL12 to trigger IFNg production of naive | 471 |
| | CD4 T cells | |
| 86_STAT4 (dimer) | regulates IL12 responses (impt for Thi diff) and mediating Th | |
| | differentiation | |
| | Pan T Cell Function | |
| 51_CCL17 | chemotactic for T cells | 23 |
| 51_THY1 | T cell surface antigen | 43 |
| 51_T cell proliferation | T cell proliferation | 55 |
| 57_alpha4/beta7 Integrin | Lymphocyte Peyer patch adhesion molecule - T cell homing | 121 |
| 11_alpha4/beta7 Integrin | Lymphocyte Peyer patch adhesion molecule - T cell homing | 122 |
| 124_alpha4/beta7 Integrin | Lymphocyte Peyer patch adhesion molecule - T cell homing | 123 |
| 84_LCK | T cell specific kinase | 317 |
| 57_alpha4/beta7 Integrin/Paxillin | Lymphocyte Peyer patch adhesion molecule - T cell homing | 333 |
| | Pro-inflammatory signaling/Innate Immunity | |
| 51_mast cell activation | mast cell activation | 2 |
| 41_RIP2/NOD2 | pro-inflammatory | 29 |
| 51_CCL26 | chemotactic for eosinphils and basophils | 35 |
| 51_CCL11 | chemotactic for eosinophils | 42 |
| 41_NEMO/A20/RIP2 | pro-inflammatory | 44 |
| 41_RIPK2 | pro-inflammatory | 45 |
| 117_RIPK2 | pro-inflammatory | 46 |
| 10_RIPK2 | pro-inflammatory | 47 |
| 4_CHUK | NFkB signaling | 137 |
| 80_IL1 alpha/IL1R1/IL1RAP/MYD88/IRAK4 | pro-inflammatory | 308 |
| 80_IL1 alpha/IL1R1/IL1RAP/MYD88 | pro-inflammatory | 348 |
| 80_IL1 alpha/IL1R1/IL1RAP | pro-inflammatory | 357 |
| 108_mol:NO | nitric oxide; pro-inflammatory | 359 |
| 80_MYD88 | pro-inflammatory | 394 |
| 80_IRAK3 | pro-inflammatory | 439 |
| 80_IL1 | pro-inflammatory | 463 |
| alpha/IL1R1/IL1RAP/MYD88/IRAK4/TOLLIP | | |
| 80_IL1A | pro-inflammatory | 498 |
| | B cell/Humoral Immunity | |
| 51_IL4 | humoral immunity/B cell differentiation | 1 |
| 51_IL13RA1 | produced by activated Th2 cells; humoral immunity | 3 |
| 32_EDN2 | B cell/humoral immunity | 4 |
| 51_IL4/IL4R/JAK1/IL13RA1/JAK2 | produced by activated Th2 cells; humoral immunity | 19 |
| 51_IL4/IL4R/JAK1/IL2R gamma/JAK3/IRS1 | produced by activated Th2 cells; humoral immunity | 20 |
| 51_IL4/IL4R/JAK1/IL2R gamma/JAK3/SHIP | produced by activated Th2 cells; humoral immunity | 21 |
| 51_T-helper 2 cell differentiation | Th2 response | 22 |
| 51_IL4/IL4R/JAK1/IL2R | produced by activated Th2 cells; humoral immunity | 24 |
| gamma/JAK3/SHC/SHIP | | |
| 51_PIGR | polymeric immunoglobulin receptor | 31 |
| 51_IL13RA2 | produced by activated Th2 cells; humoral immunity | 34 |
| 51_IL4R | humoral immunity/B cell differentiation | 36 |
| 51_IL5 | differentiation factor for B cells and eosinophils | 38 |
| 51_IGHG3 | IgG3 heavy chain | 40 |
| 51_STAT6 (dimer)/ETS1 | activated by IL4; Th2 differentiation | 50 |
| 51_STAT6 (dimer) | activated by IL4; Th2 differentiation | 51 |
| 51_STAT6 | activated by IL4; Th2 differentiation | 53 |
| 51_IL4R/JAK1 | humoral immunity/B cell differentiation | 57 |
| 51_STAT6 (dimer)/PARP14 | activated by IL4; Th2 differentiation | 58 |
| 51_IL4/IL4R/JAK1/IL2R gamma/JAK3 | humoral immunity/B cell differentiation | 62 |
| 51_IL4/IL4R/JAK1/IL2R | humoral immunity/B cell differentiation | 63 |
| gamma/JAK3/FES/IRS2 | | |
| 51_IL4/IL4R/JAK1 | humoral immunity/B cell differentiation | 64 |
| 51_IL4/IL4R/JAK1/IL2R gamma/JAK3/DOK2 | humoral immunity/B cell differentiation | 68 |
| 51_IGHG1 | IgG1 heavy chain | 74 |
| 51_STAT6 (cleaved dimer) | activated by IL4; Th2 differentiation | 75 |
| 51_FCER2 | Fc fragment of IgE receptor | 79 |
| 51_IL4/IL4R/JAK1/IL2R | humoral immunity/B cell differentiation | 101 |
| gamma/JAK3/SHC/SHIP/GRB2 | | |
| 51_IL4/IL4R/JAK1/IL2R gamma/JAK3/FES | humoral immunity/B cell differentiation | 124 |
| 22_B-cell antigen/BCR complex/LYN | B cell signaling | 209 |
| 51_IL4/IL4R/JAK1/IL2R gamma/JAK3/SHP1 | humoral immunity/B cell differentiation | 285 |
| 65_BLK | B cell tyrosine kinase | 307 |
| 22_CD72/SHP1 | B cell marker | 347 |
| 43_Fc epsilon | | |
| R1/FcgammaRIIB/SHIP/RasGAP/p62DOK | B cell signaling | 376 |
| 51_IL13RA1/JAK2 | produced by activated Th2 cells; humoral immunity | 436 |
| 51_IGHE | heavy chain of IgE | 71 |
| 51_BCL6 | regulates IL4 signaling in B cells | 494 |
| | Immunosuppression | |
| 51_IL10 | immunosuppressive cytokine | 30 |
| | Macrophage Function | |
| 110_CSF2 | Macrophage differentiation | 355 |
| 39_CSF2 | Macrophage differentiation | 469 |
| | Pan Immune Cell Function | |
| 51_LTA | cytokine produced by lymphocytes | 16 |
| 51_SELP | role in platelet activation | 33 |
| 22_DAPP1 | adaptor protein that functions within the immune system | 131 |
| 50_LEF1 | lympoid enhancer | 327 |
| 112_MEF2C/TIF2 | myocyte enhancer | 328 |
| 25_Syndecan-1/RANTES | chemotactic for macrophages and T cells | 386 |
| 22_PTPN6 | protein tyrosine phosphatase expressed within the hematopoeitic | 395 |
| | lineage | |
| 116_INPP5D | SHIP; hematopoetic specific (negatively regulates immune | 434 |
| | function) | |
| 20_VAV3 | GEF expressed in lymphoid cells | 454 |
| 86_STAT5A (dimer) | induced by many cytokines: pro-tumorigenic properties | 472 |

Table 2 lists pathway entities (individual proteins or complexes) that are located in non-immune related pathways and that are differentially regulated relative to healthy tissue these entities are from a subgroup of positive outcome patients. These entities were from a subgroup of negative outcome patients.

TABLE 2

| Chin non-immune | | Rank |
|---|---|---|
| | Cytoskeletal (actin/microtulule) | |
| 29_KIF13B | kinesin - microtubule dynamics | 398 |
| 73_SNTA1 | found in muscle fibers - microtubule dynamics | 497 |
| 37_ROCK2 | regulates actin cytoskeleton | 168 |
| 100_ROCK2 | regulates actin cytoskeleton | 273 |
| 108_PXN | regulates actin cytoskeleton | 274 |
| 103_nectin-3/I-afadin | regulates actin cytoskeleton | 275 |
| 103_nectin-3(dimer)/I-afadin/I-afadin | regulates actin cytoskeleton | 276 |
| 124_PXN | regulates actin cytoskeleton | 430 |
| | 14-3-3 signaling | |
| 4_BAD/YWHAZ | 14-3-3 signaling | 220 |
| 4_YWHAZ | 14-3-3 zeta | 10 |
| 95_YWHAZ | 14-3-3 zeta | 11 |
| 33_YWHAZ | 14-3-3 zeta | 12 |
| 46_YWHAZ | 14-3-3 zeta | 13 |
| 92_YWHAZ | 14-3-3 zeta | 14 |
| | Mitogenic response | |
| 28_MAP2K2 | activates the ERK pathway | 277 |
| 22_MAP2K1 | activates the ERK pathway | 380 |
| 28_MAPK1 | AKA: ERK1 | 401 |
| 7_MAPK8 | AKA: ERK2 | 231 |
| 51_MAPKKK cascade | MAPK signaling | 135 |
| 108_MAPKKK cascade | MAPK signaling | 346 |
| 4_MAPKKK cascade | MAPK signaling | 452 |
| 22_RAF1 | MAPK signaling | 126 |
| | stress response | |
| 108_mol:Phosphatidic acid | p38 MAPK family member | 133 |
| 95_MAP3K8 | activates ERK and JNK pathways | 219 |
| 96_MAP3K8 | activates ERK and JNK pathways | 225 |
| 42_MAP3K8 | activates ERK and JNK pathways | 228 |
| 53_MAP3K8 | activates ERK and JNK pathways | 229 |
| 93_MAP2K4 | activates JNK signaling | 349 |
| 62_MAP2K4 | activates JNK signaling | 409 |
| 27_MAP2K4 | activates JNK signaling | 470 |
| 106_MAP2K4 | activates JNK signaling | 490 |
| 7_JNK cascade | stress response | 269 |
| 4_JNK cascade | stress response | 341 |
| 106_MAPK8 | AKA: JNK1 | 423 |
| 108_MAPK8 | AKA: JNK1 | 483 |
| 51_MAPK14 | MAPK: role in stress response and cell cycle | 105 |
| 78_MAPK8 | JNK signaling | 204 |
| 51_FRAP1 | AKA: JNK1 | 100 |
| 36_ADCY3 | cAMP signaling | 397 |
| 51_BCL2L1 | adenylate cyclase | 41 |
| 51_SOCS1 | regulates PKA signaling | 15 |
| 74_mol:cAMP | cAMP signaling | 448 |
| | apoptosis | |
| 77_BIRC5 | Bcl2—apoptosis | 473 |
| 26_BIRC5 | anti-apoptotic | 118 |
| 114_BIRC5 | anti-apoptotic | 267 |
| 108_negative regulation of caspase activity | anti-apoptotic | 404 |
| 4_BAD/BCL-XL/YWHAZ | anti-apoptotic | 172 |
| 129_neuron apoptosis | apoptosis | 306 |
| 70_apoptosis | apoptosis | 493 |
| 51_ALOX15 | apoptosis | 6 |
| 28_CRADD | pro-apoptotic | 466 |
| 4_CASP9 | initiatiator caspase - apoptosis | 54 |
| 130_TRAIL/TRAILR1/DAP3/GTP | death receptor | 272 |
| 130_TRAIL/TRAILR1 | death receptor | 56 |
| 22_MAPK3 | AKA: anti-apoptotic Bcl2 family member | 406 |
| | angiogenesis | |
| 108_NOS3 | eNOS: angiogenesis | 447 |
| 108_Tie2/Ang1/GRB14 | angiogenesis | 302 |
| 108_Tie2/Ang1/ABIN2 | angiogenesis | 303 |
| 108_Tie2/Ang1/Shc | angiogenesis | 321 |
| 108_Tie2/SHP2 | angiogenesis | 323 |
| 108_vasculogenesis | angiogenesis | 334 |
| 108_Tie2/Ang1/alpha5/beta1 Integrin | angiogenesis | 345 |
| 23_angiogenesis | angiogenesis | 403 |
| 108_Tie2/Ang1 | angiogenesis | 476 |
| 2_VEGFC | angiogenesis | 115 |
| 108_response to hypoxia | hypoxic response | 453 |
| | calcium/calmodulin signaling | |
| 72_mol:Ca2+ | calcium/calmodulin signaling | 294 |
| 95_CABIN1/MEF2D/CaM/Ca2+/CAMK IV | calcium/calmodulin signaling | 332 |
| 95_CABIN1/YWHAQ/CaM/Ca2+/CAMK IV | calcium/calmodulin signaling | 283 |
| 117_PRKACB | cAMP dependent protein kinase | 103 |
| | Cell cycle | |
| 15_PLK2 | cell cycle | 337 |
| 15_PLK2 | cell cycle | 309 |
| 40_MNAT1 | cell cycle | 304 |
| 114_CDK4 | cell cycle/G1-S | 130 |
| 112_CDK4 | cell cycle/G1-S | 316 |
| 110_E2F1 | cell cycle/G1-S | 495 |
| 110_CDK4 | cell cycle/G1-S | 73 |
| 100_CDC2 | cell cycle/mitosis | 87 |
| 100_CCNB1 | cell cycle/mitosis | 95 |
| 51_mitosis | cell cycle/mitosis | 111 |
| 90_INCENP | cell cycle/mitosis | 112 |
| 100_INCENP | cell cycle/mitosis | 113 |
| 77_INCENP | cell cycle/mitosis | 195 |
| 77_mitotic metaphase/anaphase transition | cell cycle/mitosis | 197 |
| 120_NDEL1 | cell cycle/mitosis | 208 |
| 47_regulation of S phase of mitotic cell cycle | cell cycle/mitosis | 354 |
| 77_CDCA8 | cell cycle/mitosis | 393 |
| 100_SPC24 | cell cycle/mitosis | 396 |
| 26_NDEL1 | cell cycle/mitosis | 419 |
| 15_regulation of centriole replication | cell cycle/mitosis | 456 |
| 100_CCNB1/CDK1 | cell cycle/mitosis | 491 |
| 77_Chromosomal passenger complex | cell cycle/mitosis | 479 |
| 74_positive regulation of cyclin-dependent protein | cell cycle | 261 |
| kinase activity | | |
| 123_TIMELESS/CRY2 | cell cycle/S phase | 440 |
| 77_EVI5 | cell cycle; G1-S | 27 |
| | chromatin remodeling | |
| 47_KAT2B | lysine acetyltransferase; histone modification | 97 |
| 52_Histones | histone | 207 |
| 47_HIST2H4A | histone | 117 |
| 52_HDAC6/HDAC11 | histone deacetylase | 139 |
| 52_HDAC11 | histone deacetylase | 290 |
| 52_HDAC5/BCL6/BCoR | histone deacetylase | 363 |
| 63_HDAC1/Smad7 | histone deacetylase | 364 |
| 66_HDAC2 | histone deacetylase | 405 |
| 50_HDAC1 | histone deacetylase | 425 |
| 52_HDAC5/RFXANK | histone deacetylase | 402 |
| 52_positive regulation of chromatin silencing | chromatin remodeling | 106 |
| 47_SIRT1/MEF2D/HDAC4 | chromatin remodeling | 184 |
| 61_SIRT1 | chromatin remodeling | 185 |
| 106_SIRT1 | chromatin remodeling | 192 |
| 47_SIRT1/p300 | chromatin remodeling | 193 |
| 47_KU70/SIRT1 | chromatin remodeling | 214 |
| 47_SIRT1 | chromatin remodeling | 442 |
| 106_NCOA1 | chromatin remodeling | 165 |
| | ECM | |
| 23_FN1 | fibronectin - ECM | 292 |
| 25_LAMA5 | laminin 5 - ECM | 420 |
| 64_LAMA3 | laminin 5 - ECM | 421 |
| 78_LAMA3 | laminin 5 - ECM | 377 |
| 51_COL1A1 | collagen 1 A1 - ECM | 66 |
| 51_COL1A2 | collagen 1 A2 - ECM | 362 |
| 112_COL1A2 | collagen 1 A2 - ECM | 218 |
| | DNA damage response | |
| 100_BUB1 | DNA damage response | 173 |
| 13_PRKDC | DNA damage response | 196 |
| 77_BUB1 | DNA damage response | 202 |
| 49_RAD50 | DNA damage response | 203 |
| 30_RAD50 | DNA damage response | 210 |
| 4_PRKDC | DNA damage response | 211 |
| 49_PRKDC | DNA damage response | 230 |
| 20_PRKDC | DNA damage response | 300 |
| 40_TFIIH | DNA damage response | 305 |
| 49_DNA-PK | DNA damage response | 311 |
| 49_BARD1/DNA-PK | DNA damage response | 319 |
| 20_DNA-PK | DNA damage response | 329 |
| 49_FANCE | DNA damage response | 338 |
| 49_FANCA | DNA damage response | 435 |
| 30_ATM | DNA damage response | 437 |
| 30_DNA damage response signal transduction by p53 | DNA damage response | 413 |
| class mediator resulting in induction of apoptosis | | |
| | PLC Signaling | |
| 79_PLCB1 | phospholipase C b1 | 142 |
| 108_PLD2 | phospholipase D2 | 186 |
| 72_PLCG1 | phospholipase G1 | 120 |
| | PKC signaling | |
| 95_PRKCH | protein kinase C-eta (epithelial specifc) | 94 |
| 78_GO:0007205 | PKC signaling | 157 |
| 72_mol:DAG | PKC signaling | 158 |
| 72_mol:IP3 | PKC signaling | 291 |
| 43_calcium-dependent protein kinase C activity | PKC signaling | 313 |
| 98_PTP4A2 | RTK signaling | |
| 124_PTK2 | FAK family member | 25 |
| 108_PTK2 | FAK family member | 312 |
| 104_FRS3 | FGFR substrate | 465 |
| | RTK signaling | 299 |
| 81_EPHA5 | RTK signaling | 119 |
| 108_TEK | RTK signaling | 160 |
| 19_Ephrin B1/EPHB3 | protein tyrosine phosphatase | 164 |
| 77_RACGAP1 | RTK signaling | 287 |
| 104_SHC/RasGAP | RTK signaling | 174 |
| 19_EPHB3 | RTK signaling | 175 |
| 117_proNGF (dimer)/p75(NTR)/Sortilin/MAGE-G1 | RTK signaling | 177 |
| 65_GPC1/NRG | RTK signaling | 178 |
| 108_Crk/Dok-R | RTK signaling | 189 |
| 65_NRG1 | RTK signaling | 190 |
| 87_NRG1 | RTK signaling | 200 |
| 7_RET51/GFRalpha1/GDNF/DOK/RasGAP/NCK | RTK signaling | 213 |
| 94_SOS1 | RTK signaling | 217 |
| 72_E6FR/PI3K-beta/Gab1 | RTK signaling | 226 |
| 17_NRG1 | RTK signaling | 288 |
| 91_PDGFB-D/PDGFRB/APS/CBL | RTK signaling | 367 |
| 7_RET9/GFRalpha1/GDNF/SHC | RTK signaling | 368 |
| 7_RET51/GFRalpha1/GDNF/SHC | RTK signaling | 369 |
| 7_RET9/GFRalpha1/GDNF/Shank3 | RTK signaling | 370 |
| 7_RET51/GFRalpha1/GDNF/FRS2 | RTK signaling | 371 |
| 7_RET9/GFRalpha1/GDNF/FRS2 | RTK signaling | 372 |
| 7_RET51/GFRalpha1/GDNF/GRB10 | RTK signaling | 373 |
| 7_RET9/GFRalpha1/GDNF/IRS1 | RTK signaling | 374 |
| 7_RET51/GFRalpha1/GDNF/DOK1 | RTK signaling | 375 |
| 7_RET51/GFRalpha1/GDNF/IRS1 | RTK signaling | 381 |
| 19_Ephrin B/EPHB2/RasGAP | RTK signaling | 389 |
| 7_RET9/GFRalpha1/GDNF | RTK signaling | 422 |
| 116_LYN/PLCgamma2 | RTK signaling | 426 |
| 17_ErbB4/ErbB4/neuregulin 1 beta/neuregulin 1 | RTK signaling | 427 |
| beta/Fyn | | |
| 17_ErbB4/EGFR/neuregulin 1 beta | RTK signaling | 438 |
| 17_ErbB4 CYT2/ErbB4 CYT2/neuregulin 1 | tyrosine kinase | 26 |
| beta/neuregulin 1 beta | | |
| 30_ABL1 | tyrosine kinase | 49 |
| 84_FER | tyrosine kinase | 485 |
| 108_BMX | tyrosine phosphorylation of Cb1 | 296 |
| 88_SORBS1 | RTK signaling | 492 |
| 13_MET | adaptor protein | 61 |
| 72_GAB1 | adaptor protein | 156 |
| 7_GRB10 | adaptor protein | 314 |
| 108_NCK1/Dok-R | Src family kinase | 280 |
| 84_FYN | Src family kinase | 298 |
| 43_FYN | Src family member | 310 |
| 65_HCK | ser/thr phosphatase | 128 |
| 22_PPP3CC | ser/thr phosphatase | 199 |
| 25_PPIB | ser/thr phosphatase | 353 |
| 100_PPP2R1A | ser/thr phosphatase | 412 |
| 100_PP2A-alpha B56 | ser/thr phosphatase | |
| 51_mol:PI-3-4-5-P3 | PI3K/AKT signaling | 99 |
| 51_AKT1 | signaling/pro-survival | 102 |
| 51_PI3K | signaling/pro-survival | 109 |
| 4_TSC1 | downstream negative regulator of AKT | 69 |
| 74_PIK3R1 | signaling/pro-survival | 205 |
| 55_PIK3R1 | signaling/pro-survival | 212 |
| 108_PIK3R1 | signaling/pro-survival | 215 |
| 9_PIK3R1 | signaling/pro-survival | 221 |
| 38_PIK3R1 | signaling/pro-survival | 223 |
| 72_PIK3R1 | signaling/pro-survival | 227 |
| 43_PIK3R1 | signaling/pro-survival | 232 |
| 103_PIK3R1 | signaling/pro-survival | 233 |
| 2_PIK3R1 | signaling/pro-survival | 234 |
| 23_PIK3R1 | signaling/pro-survival | 235 |
| 88_PIK3R1 | signaling/pro-survival | 236 |
| 101_PIK3R1 | signaling/pro-survival | 237 |
| 104_PIK3R1 | signaling/pro-survival | 238 |
| 79_PIK3R1 | signaling/pro-survival | 239 |
| 51_PIK3R1 | signaling/pro-survival | 240 |
| 109_PIK3R1 | signaling/pro-survival | 241 |
| 117_PIK3R1 | signaling/pro-survival | 242 |
| 124_PIK3R1 | signaling/pro-survival | 243 |
| 7_PIK3R1 | signaling/pro-survival | 244 |
| 113_PIK3R1 | signaling/pro-survival | 245 |
| 69_PIK3R1 | signaling/pro-survival | 246 |
| 116_PIK3R1 | signaling/pro-survival | 247 |
| 119_PIK3R1 | signaling/pro-survival | 248 |
| 131_PIK3R1 | signaling/pro-survival | 249 |
| 80_PIK3R1 | signaling/pro-survival | 250 |
| 91_PIK3R1 | signaling/pro-survival | 251 |
| 135_PIK3R1 | signaling/pro-survival | 252 |
| 68_PIK3R1 | signaling/pro-survival | 253 |
| 84_PIK3R1 | signaling/pro-survival | 254 |
| 46_PIK3R1 | signaling/pro-survival | 255 |
| 3_PIK3R1 | signaling/pro-survival | 256 |
| 57_PIK3R1 | signaling/pro-survival | 257 |
| 19_PIK3R1 | signaling/pro-survival | 258 |
| 45_PIK3R1 | signaling/pro-survival | 259 |
| 22_PIK3R1 | signaling/pro-survival | 260 |
| 70_PIK3R1 | signaling/pro-survival | 262 |
| 94_PIK3R1 | signaling/pro-survival | 263 |
| 93_PIK3R1 | signaling/pro-survival | 266 |
| 122_PIK3R1 | signaling/pro-survival | 268 |
| 72_mol:PIP3 | signaling/pro-survival | 279 |
| 4_AKT1 | signaling/pro-survival | 330 |
| 4_AKT1/RAF1 | signaling/pro-survival | 335 |
| 4_AKT1/ASK1 | signaling/pro-survival | 339 |
| 108_AKT1 | signaling/pro-survival | 445 |
| 108_PI3K | signaling/pro-survival | 475 |
| 51_RPS6KB1 | signaling/pro-survival | 141 |
| 4_mTOR/RHEB/GDP/Raptor/GBL/PRAS40 | ribosomal protein S6 kinase - signaling | 384 |
| 74_SMPD1 | signaling/translational control | 270 |
| 4_AKT1S1 | AKA:mTOR - signaling | 366 |
| 44_NDRG1 | AKT substrate | 342 |
| | sphingosine 1 phosphate | |
| 83_S1P/S1P3/Gq | sphingomyelinase; generates ceramide | 159 |
| 112_SP1 | sphingosine 1 phosphate | 224 |
| 1_S1P/S1P5/G12 | sphingosine 1 phosphate | 338 |
| 1_mol:S1P | sphingosine 1 phosphate | 337 |
| 61_SP1 | sphingosine 1 phosphate | 265 |
| 1_S1P/S1P3/Gq | sphingosine 1 phosphate | 315 |
| 51_SP1 | sphingosine 1 phosphate | 487 |
| 14_SP1 | sphingosine 1 phosphate | 488 |
| 44_SP1 | sphingosine 1 phosphate | 489 |
| 51_JAK1 | sphingosine 1 phosphate | 5 |
| 105_BAMBI | TGFb signaling | 5 |
| 65_TGFBR1 (dimer) | TGFb signaling | 104 |
| 105_BMP2-4/BMPR2/BMPR1A- | TGFb signaling | 162 |
| 1B/RGM/ENDOFIN/GADD34/PP1CA | | |
| 65_GPC1/TGFB/TGFBR1/TGFBR2 | TGFb signaling | 180 |
| 23_TGFBR2 | TGFb signaling | 181 |
| 65_TGFBR2 | TGFb signaling | 182 |
| 65_TGFBR2 (dimer) | TGFb signaling | 183 |
| 105_BMP2-4/BMPR2/BMPR1A-1B/RGM/XIAP | TGFb signaling | 326 |
| 105_SMAD7/SMURF1 | TGFb signaling | 350 |
| 105_SMAD7 | TGFb signaling | 443 |
| 63_SMAD7 | TGFb signaling | 444 |
| 105_BMPR2 (homodimer) | TGFb signaling | 474 |
| | TGFb signaling | |
| 56_JAM3 | cell adhesion | 410 |
| 78_positive regulation of cell-cell adhesion | cell adhesion | 343 |
| 23_cell adhesion | cell adhesion | 309 |
| 51_ITGB3 | integrin beta 3 | 88 |
| 11_ITGB7 | integrin beta 7 | 89 |
| 124_ITGB7 | integrin beta 7 | 90 |
| 45_ITGB7 | integrin beta 7 | 91 |
| 57_ITGB7 | integrin beta 7 | 179 |
| 56_JAM3 homodimer | tight junctional protein | 411 |
| | tight junctional protein | |
| 47_FOXO3 | Transcription factor | 7 |
| 47_FOXO1/FHL2/SIRT1 | transcription factor | 110 |
| 47_SIRT1/FOXO3a | transcription factor | 116 |
| 123_NPAS2 | transcription factor | 166 |
| 106_JUN | transcription factor | 222 |
| 7_JUN | transcription factor | 271 |
| 126_MYC | transcription factor | 318 |
| 108_FOXO1 | transcription factor | 356 |
| 50_MYC | transcription factor | 379 |
| 92_FOXO3A/14-3-3 | transcription factor | 382 |
| 75_NFAT1/CK1 alpha | transcription factor | 383 |
| 4_FOXO1-3 a-4/14-3-3 family | transcription factor | 408 |
| 4_FOXO1 | transcription factor | 415 |
| 4_FOXO3 | transcription factor | 416 |
| 4_FOXO4 | transcription factor | 417 |
| 113_AP1 | transcription factor | 432 |
| 30_MYC | transcription factor | 449 |
| 50_HNF1A | transcription factor | 486 |
| 20_PATZ1 | transcription factor | 499 |
| 51_EGR2 | transcription factor | 52 |
| | transcription factor; regulates ErbB2 exspression | |
| 72_GNA11 | G protein signaling | 78 |
| 33_mol:GTP | GTP function | 281 |
| 16_mol:GDP | GTP function | 295 |
| 72_mol:GTP | GTP function | 322 |
| 24_Gi family/GNB1/GNG2/GDP | GTP function | 309 |
| 4_mol:GDP | GTP function | 481 |
| 63_mol:GTP | GTP function | 28 |
| 79_GNB1/GNG2 | G protein | 385 |
| 97_Rac/GTP | G protein - cell motility | 191 |
| 32_EntrezGene:2778 | G protein signaling | 428 |
| 58_GNB1 | G regulatory protein function | 496 |
| 24_GNB1 | G regulatory protein function | 451 |
| 29_CENTA1/KIF3B | ARF protein - trafficking | 216 |
| 1_ABCC1 | ARF-GAP | 458 |
| 14_NF1 | negatively regulates Ras pathway | 477 |
| 78_NF1 | negatively regulates Ras pathway | 478 |
| 135_NF1 | negatively regulates Ras pathway | 92 |
| 116_RAPGEF1 | Rac GAP protein | 188 |
| 7_HRAS/GTP | RAP GEF | 441 |
| 5_RAN | Ras family member | 324 |
| 63_RAN | Ras family member/nucleocytoplasmic transport | 351 |
| 97_ARF1/GTP | Ras family member/nucleocytoplasmic transport | 169 |
| 108_RasGAP/Dok-R | Ras family member/protein trafficking | 127 |
| 43_RasGAP/p62DOK | Ras signaling | 390 |
| 108_RASA1 | RasGAP | 143 |
| 19_RASA1 | Ras-GAP | 144 |
| 109_RASA1 | Ras-GAP | 145 |
| 78_RASA1 | Ras-GAP | 146 |
| 43_RASA1 | Ras-GAP | 147 |
| 77_RASA1 | Ras-GAP | 148 |
| 88_RASA1 | Ras-GAP | 149 |
| 7_RASA1 | Ras-GAP | 150 |
| 26_RASA1 | Ras-GAP | 151 |
| 104_RASA1 | Ras-GAP | 152 |
| 22_RASA1 | Ras-GAP | 153 |
| 92_SOD2 | Ras-GAP | 457 |
| 29_GNA11 | trimeric G protein | 82 |
| 1_GNA11 | trimeric G protein | 83 |
| 83_GNA11 | trimeric G protein | 84 |
| 58_GNA11 | trimeric G protein | 85 |
| 79_GNA11 | trimeric G protein | 86 |
| 32_GNA11 | trimeric G protein | 93 |
| 58_Gq family/GTP | trimeric G protein | 114 |
| 79_Gq family/GTP | trimeric G protein | 140 |
| 58_Gq family/GTP/EBP50 | trimeric G protein | 194 |
| 79_Gq family/GDP/Gbeta gamma | trimeric G protein | 278 |
| 1_GNA12 | trimeric G protein | 336 |
| 89_GNAT1 | trimeric G protein | 407 |
| 19_PAK1 | trimeric G protein | 198 |
| 88_TC10/GDP | Rho effector kinase | 167 |
| 103_CDC42 | Rho family member; cell motility | 289 |
| 33_RHOQ | Rho family member; cell motility | 467 |
| 59_ARHGEF6 | Rho family member; cell motility | 399 |
| 19_KALRN | Rho GEF | 365 |
| | Rho GEF kinase | |
| | Ubiquitination | 284 |
| 77_Chromosomal passenger complex/Cul3 protein | ubiquitinitation | 361 |
| complex | | |
| 63_ubiquitin-dependent protein catabolic process | ubiquitinitation | 107 |
| 133_MDM2 | ubiquitinitation of p53 | 59 |
| 51_CBL | ubiquitinitation of RTKs | |
| | metabolism | |
| 47_ACSS2 | acyl CoA synthetase | 206 |
| 52_NPC | cholesterol trafficking | 134 |
| 44_PFKFB3 | glucose metabolism | 378 |
| 47_SIRT1/PGC1A | metabolism | 358 |
| 108_mol:NADP | metabolism | 360 |
| 108_mol:L-citrulline | metabolism | 446 |
| 123_mol:NADPH | metabolism | 297 |
| | Other | 482 |
| 51_AICDA | activation-induced cytidine deaminase | 81 |
| | alpha/beta hydrolase | 301 |
| 129_APP | amyloid beta precursor protein | 461 |
| 117_APP | amyloid beta precursor protein | 462 |
| 65_APP | amyloid beta precursor protein | 98 |
| 125_ARF1 | arachidonate 15-lipoxygenase | 418 |
| 82_ABCC1 | ATP transporter; multi drug resistance | 460 |
| 4_BAD/BCL-XL | ATP transporter; multi drug resistance | 424 |
| 127_mol:Bile acids | bile acid | 201 |
| 56_PLAT | blood coagulation | 387 |
| 88_F2RL2 | blood coagulation | 484 |
| 108_PLG | blood coagulation | 136 |
| 37_bone resorption | bone remodeling | 163 |
| 123_mol:CO | carbon monoxide | 154 |
| 86_JAK1 | stat signaling | 310 |
| 92_GADD45A | cell cycle arrest and apoptosis (p53 inducible) | 80 |
| 51_JAK2 | stat signaling | 336 |
| 109_cell morphogenesis | cell shape | 155 |
| 78_Syndecan-2/Syntenin/PI-4-5-P2 | cell surface proteoglycan | 108 |
| 108_mol:Choline | choline | 72 |
| 123_CLOCK | circadian rythym | 67 |
| 5_EntrezGene:9972 | component of the nuclear pore complex | 282 |
| 5_EntrezGene:23636 | component of the nuclear pore complex | 161 |
| 44_EDN1 | endothelin 1 - vasoconstriction | 400 |
| 123_mol:HEME | erythropoeisis | 450 |
| 79_ESR1 | estrogen signaling | 96 |
| 131_GRN2B | glutamate receptor | 459 |
| 17_GRIN2B | glutamate receptor | 264 |
| 89_GUCA1A | guanylate cyclase | 433 |
| 20_PIAS3 | inhibits Stat signaling | 414 |
| 24_IFT88 | intraflagellar transport | 331 |
| 20_FHL2 | LIM domain containing protein | 325 |
| 23_MFGE8 | milk fat globule-EGF factor 8 protein | 500 |
| 20_HNRNPA1 | mRNA processing | 76 |
| 47_muscle cell differentiation | muscle cell differentiation | 77 |
| 47_SIRT1/PCAF/MYOD | muscle cell differentiation | 429 |
| 105_RGMB | neuronal function | 132 |
| 19_neuron projection morphogenesis | neuronal function | 176 |
| 65_neuron differentiation | neuronal function | 391 |
| 7_GFRalpha1/GDNF | neurotrophic receptor | 32 |
| 51_OPRM1 | opioid receptor | 171 |
| 85_hyperosmotic response | osmosis | 455 |
| 79_MAPK11 | phosphatidic acid | 187 |
| 89_PDE6G/GNAT1/GTP | phosphodiesterase | 344 |
| 84_Prolactin Receptor/Prolactin | pregnancy hormone | 340 |
| 17_Prolactin receptor/Prolactin receptor/Prolactin | pregnancy hormone | 464 |
| 78_TRAPPC4 | protein trafficking | 37 |
| 27_MAP3K12 | reactive oxygen species | 480 |
| 51_SOCS3 | regulates Stat signaling | 70 |
| 51_SOCS5 | regulates Stat signaling | 129 |
| 51_RETNLB | regulates Stat signaling | 60 |
| 40_CRBP1/9-cic-RA | resistin like beta | 9 |
| 40_RBP1 | retinol binding protein | 17 |
| 51_TFF3 | secreted protein normally found in the GI mucosa | 65 |
| 68_DHH N/PTCH1 | sonic hedgehog receptor | |
| 74_EIF3A | translation | 468 |
| 78_Syndecan-2/CASK/Protein 4.1 | transmembrane proteoglycan | 48 |
| 66_VIPR1 | vasoconstriction | 293 |
| 32_ETB receptor/Endothelin-3 | vasoconstriction | 320 |
| 45_E-cadherin/Ca2+/beta catenin/alpha catenin | Wnt signaling | 18 |

Table 3 lists pathway entities (individual proteins or complexes) that are located in immune related pathways and that are differentially regulated relative to healthy tissue. These entities were from a subgroup of positive outcome patients.

TABLE 3

| MicMa Immune-Related | Function | Rank |
|---|---|---|
| PathwayEntity | Anti-tumor Immunity (NK cell, CTL, M1 macrophage function) | |
| 86_IL12B | important for Th1 differentiation | 18 |
| 51_T-helper 1 cell differentiation | important for Th1 differentiation | 35 |
| 9_IL12B | important for Th1 differentiation | 55 |
| 10_IL12B | important for Th1 differentiation | 144 |
| 86_IFNG | anti-tumor immunity | 145 |
| 77_PSMA3 | immunoproteasome | 203 |
| 39_IFNG | anti-tumor immunity | 403 |
| | Pan T Cell Function | |
| 51_T cell proliferation | T cell proliferation | 6 |
| 51_THY1 | T cell surface antigen | 9 |
| 51_CCL17 | chemotactic for T cells | 70 |
| 95_PRKCQ | PKC theta - important for T cell activation | 178 |
| 110_PRKCQ | PKC theta - important for T cell activation | 179 |
| 114_NFATC3 | nuclear factor of activated T cells | 210 |
| 42_EntrezGene:6957 | TCR beta | 385 |
| 39_NFATC2 | nuclear factor of activated T cells | 458 |
| | Pro-inflammatory signaling/Innate Immunity | |
| 51_CCL11 | chemotactic for eosinophils | 12 |
| 51_CCL26 | chemotactic for eosinphils and basophils | 17 |
| 30_IFNAR2 | IFN alpha/beta receptor - proinflammatory | 25 |
| 80_SQSTM1 | regulates NFkB activation - inflammatory | 26 |
| 104_SQSTM1 | regulates NFkB activation - inflammatory | 27 |
| 117_SQSTM1 | regulates NFkB activation - inflammatory | 28 |
| 80_IRAK4 | activates NFkB - inflammatory | 37 |
| 12_NFKBIA | pro-inflammatory | 59 |
| 28_NFKBIA | pro-inflammatory | 120 |
| 118_NFKBIA | pro-inflammatory | 121 |
| 93_IL6ST | pro-inflammatory | 168 |
| 9_NFKBIA | pro-inflammatory | 175 |
| 86_IL6ST | pro-inflammatory | 206 |
| 85_MAP3K1 | binds TRAF2; stimulates NFkB | 231 |
| 95_MAP3K1 | binds TRAF2; stimulates NFkB | 232 |
| 115_MAP3K1 | binds TRAF2; stimulates NFkB | 233 |
| 30_IRF1 | activates IFN alpha and beta transcription - inflammatory | 343 |
| 70_IRF9 | IFN alpha responsive gene - inflammatory | 345 |
| 41_NFKBIA | pro-inflammatory | 358 |
| 2_MAP3K13 | binds TRAF2; stimulates NFkB | 409 |
| 63_NFKBIA | pro-inflammatory | 452 |
| 16_PTGS2 | prostaglandin synthase - proinflammatory | 487 |
| 30_IFN-gamma/IRF1 | activates IFN alpha and beta transcription - inflammatory | 488 |
| | B cell/Humoral Immunity | |
| 51_IL4 | B cell/humoral immunity | 1 |
| 51_IL5 | differentiation factor for B cells (eosinophils) | 3 |
| 51_STAT6 (cleaved dimer) | activated by IL4; Th2 differentiation | 7 |
| 51_IGHG3 | heavy chain of IgG3 | 8 |
| 51_IL4R | B cell/humoral immunity | 10 |
| 51_IL13RA2 | B cell/humoral immunity | 11 |
| 51_STAT6 (dimer)/PARP14 | activated by IL4; Th2 differentiation | 13 |
| 51_IL4/IL4R/JAK1 | B cell/humoral immunity | 16 |
| 51_IL4R/JAK1 | B cell/humoral immunity | 44 |
| 51_PIGR | polymeric immunoglobulin receptor | 96 |
| 51_IL13RA1 | B cell/humoral immunity | 100 |
| 110_T-helper 2 cell lineage commitment | B cell/humoral immunity | 111 |
| 51_STAT6 (dimer)/ETS1 | activated by IL4; Th2 differentiation | 142 |
| 10_IL4 | B cell/humoral immunity | 155 |
| 22_PI3K/BCAP/CD19 | B cell marker | 165 |
| 51_T-helper 2 cell differentiation | B cell/humoral immunity | 170 |
| 51_IL4/IL4R/JAK1/IL2R | B cell/humoral immunity | 171 |
| gamma/JAK3/DOK2 | | |
| 51_STAT6 | activated by IL4; Th2 differentiation | 176 |
| 51_STAT6 (dimer) | activated by IL4; Th2 differentiation | 189 |
| 51_IL4/IL4R/JAK1/IL2R | B cell/humoral immunity | 190 |
| gamma/JAK3/SHIP | | |
| 51_FCER2 | Fc fragment of IgE receptor | 194 |
| 51_IL4/IL4R/JAK1/IL13RA1/JAK2 | B cell/humoral immunity | 195 |
| 51_IL4/IL4R/JAK1/IL2R | B cell/humoral immunity | 207 |
| gamma/JAK3/SHC/SHIP | | |
| 51_IL4/IL4R/JAK1/IL2R | B cell/humoral immunity | 230 |
| gamma/JAK3/FES/IRS2 | | |
| 51_IL4/IL4R/JAK1/IL2R gamma/JAK3 | B cell/humoral immunity | 236 |
| 51_IL4/IL4R/JAK1/IL2R | B cell/humoral immunity | 280 |
| gamma/JAK3/SHC/SHIP/GRB2 | | |
| 51_IL4/IL4R/JAK1/IL2R | B cell/humoral immunity | 315 |
| gamma/JAK3/IRS1 | | |
| 51_IL4/IL4R/JAK1/IL2R | B cell/humoral immunity | 316 |
| gamma/JAK3/FES | | |
| 51_IL4/IL4R/JAK1/IL2R | B cell/humoral immunity | 319 |
| gamma/JAK3/SHP1 | | |
| 112_IGHV3OR16-13 | Ig variable chain | 356 |
| 39_IL4 | B cell/humoral immunity | 386 |
| 51_IGHG1 | IgG1 heavy chain | 401 |
| | Immunosuppression | |
| 51_IL10 | immunosuppressive cytokine | 43 |
| | Macrophage Function | |
| 42_PRKCE | protein kinase C-epsilon-impt for LPS-mediated function in M1 | 342 |
| | macrophage | |
| 84_CSF1R | macrophage differentiation | 445 |
| 51_ARG1 | M2 macrophage marker | 447 |
| | Pan Immune Cell Function | |
| 51_LTA | cytokine produced by lymphocytes | 15 |
| 51_SELP | role in platelet activation | 58 |
| 63_FKBP3 | protein folding; immunoregulation | 62 |
| 94_STAT5A (dimer) | induced by many cytokines; pro-tumorigenic properties | 450 |
| 53_LCP2 | lymphocyte specific adaptor protein | 456 |
| 43_LCP2 | lymphocyte specific adaptor protein | 457 |
| 42_LCP2 | lymphocyte specific adaptor protein | 459 |
| 108_DOK2 | adaptor protein expressed in hematopoeitic progenitors | 492 |
| 51_DOK2 | adaptor protein expressed in hematopoeitic progenitors | 493 |
| 62_platelet activation | platelet function | 243 |

Table 4 lists pathway entities (individual proteins or complexes) that are located in non-immune related pathways and that are differentially regulated relative to healthy tissue these entities are from a subgroup of positive outcome patients. These entities were from a subgroup of positive outcome patients.

| MicMa (non-immune) | | Rank |
|---|---|---|
| | Cytoskeletal (actin/microtulule) | |
| 45_actin cytoskeleton organization | actin dynamics | 254 |
| 131_MAPT | AKA: Tau - microtubule associated protein | 204 |
| 120_DYNC1H1 | dynein - microtubule dynamics | 331 |
| 24_KIF3A | kinesin; microtubule dynamics | 123 |
| 77_KIF2C | kinesin; microtubule dynamics | 159 |
| 100_KIF2A | kinesin; microtubule dynamics | 369 |
| 100_positive regulation of microtubule | microtubule dynamics | 367 |
| depolymerization | | |
| 73_STMN1 | microtubule dynamics | 451 |
| | Mitogenic signaling | |
| 32_MAP2K1 | activates ERK pathway | 477 |
| 87_MAPK3 | AKA: ERK1 | 443 |
| 40_MAPK1 | AKA: ERK2 | 31 |
| 115_MAPK1 | AKA: ERK2 | 32 |
| 126_MAPK1 | AKA: ERK2 | 33 |
| 105_MAPK1 | AKA: ERK2 | 34 |
| 66_MAPK1 | AKA: ERK2 | 38 |
| 62_MAPK1 | AKA: ERK2 | 182 |
| 98_MAPK1 | AKA: ERK2 | 225 |
| 27_DUSP1 | dual specificity phosphatase; suppresses MAPK | 317 |
| 43_DUSP1 | dual specificity phosphatase; suppresses MAPK | 318 |
| | Stress signaling | |
| 19_MAP4K4 | activates JNK pathway | 467 |
| 2_MAP2K3 | activates p38MAPK - stress signaling | 413 |
| 95_MAPK14 | MAPK: role in stress response and cell cycle | 193 |
| 69_MAPK14 | MAPK: role in stress response and cell cycle | 200 |
| 40_MAPK14 | MAPK: role in stress response and cell cycle | 201 |
| 85_MAPK14 | MAPK: role in stress response and cell cycle | 202 |
| 66_MAPK14 | MAPK: role in stress response and cell cycle | 226 |
| 16_MAPK14 | MAPK: role in stress response and cell cycle | 240 |
| 67_MAPK14 | MAPK: role in stress response and cell cycle | 373 |
| 51_MAPK14 | MAPK: role in stress response and cell cycle | 375 |
| 51_MAPKKK cascade | regulates JNK and ERK pathways | 213 |
| 19_JNK cascade | JNK signaling | 473 |
| | Angiogenesis | |
| 2_VEGFR2 homodimer/VEGFA | angiogenesis | 408 |
| homodimer/GRB10/NEDD4 | | |
| 2_VEGFR2 homodimer/VEGFA | angiogenesis | 415 |
| homodimer/alphaV beta3 Integrin | | |
| 2_VEGFR2 homodimer/VEGFA | angiogenesis | 475 |
| homodimer | | |
| 2_NRP2 | regulates angiogenesis | 198 |
| 3_NRP2 | regulates angiogenesis | 199 |
| 44_HIF1A | hypoxic response | 140 |
| 23_EDIL3 | integrin ligand; role in angiogenesis | 101 |
| 108_blood circulation | hemovascular | 235 |
| | Apoptosis | |
| 114_BIRC5 | anti-apoptotic function | 172 |
| 130_TNFRSF10C | anti-apoptotic function | 314 |
| 23_apoptosis | apoptosis | 219 |
| 51_BCL2L1 | AKA: anti-apoptotic Bcl2 family member | 20 |
| 130_TRAILR3 (trimer) | pro-apoptotic | 313 |
| 39_FASLG | Fas ligand - pro-apoptotic | 391 |
| | Nuclear Hormone Receptor | |
| 106_ZMIZ2 | binds nuclear hormone receptors | 417 |
| 127_PPARD | nuclear hormone receptor | 23 |
| 126_PPARD | nuclear hormone receptor | 24 |
| 40_RAR alpha/9cRA/Cyclin H | nuclear hormone receptor | 137 |
| 40_RAR alpha/9cRA | nuclear hormone receptor | 205 |
| 52_NR3C1 | nuclear hormone receptor | 334 |
| 106_NR3C1 | nuclear hormone receptor | 335 |
| 112_NR3C1 | nuclear hormone receptor | 351 |
| 52_Glucocorticoid | nuclear hormone receptor | 399 |
| receptor/Hsp90/HDAC6 | | |
| 40_RXRA | nuclear hormone receptor | 400 |
| | Calcium/Calmodulin signaling | |
| 95_CALM1 | calmodulin | 61 |
| 70_CALM1 | calmodulin | 71 |
| 3_CALM1 | calmodulin | 83 |
| 85_CALM1 | calmodulin | 84 |
| 120_CALM1 | calmodulin | 85 |
| 62_CALM1 | calmodulin | 86 |
| 33_CALM1 | calmodulin | 87 |
| 115_CALM1 | calmodulin | 88 |
| 74_CALM1 | calmodulin | 89 |
| 2_CALM1 | calmodulin | 90 |
| 39_CALM1 | calmodulin | 99 |
| 95_CaM/Ca2+/Calcineurin A alpha-beta | calmodulin | 117 |
| B1 | | |
| 95_CaM/Ca2+ | calmodulin | 118 |
| 33_AS160/CaM/Ca2+ | calmodulin | 129 |
| 33_CaM/Ca2+ | calmodulin | 130 |
| 120_CaM/Ca2+ | calmodulin | 131 |
| 51_mast cell activation | calmodulin | 133 |
| 95_CaM/Ca2+/CAMK IV | calmodulin | 160 |
| 39_CaM/Ca2+ | calmodulin | 162 |
| 39_CaM/Ca2+/Calcineurin A alpha-beta | calmodulin | 164 |
| B1 | | |
| 110_CALM1 | calmodulin | 188 |
| 110_CaM/Ca2+/Calcineurin A alpha- | calmodulin | 424 |
| beta B1 | | |
| 3_CaM/Ca2+ | calmodulin | 489 |
| 52_CAMK4 | calmodulin signaling | 270 |
| 95_CAMK4 | calmodulin signaling | 271 |
| | cAMP signaling | |
| 16_CREB1 | cAMP response element | 158 |
| 112_CREB1 | cAMP response element | 402 |
| 62_mol:cAMP | cAMP signaling | 252 |
| 95_AKAP5 | PKA signaling | 344 |
| | Casein kinase | |
| 95_CSNK1A1 | casein kinase 1, alpha 1 | 93 |
| 92_CSNK1A1 | casein kinase 1, alpha 1 | 125 |
| 75_CSNK1A1 | casein kinase 1, alpha 1 | 126 |
| 24_CSNK1A1 | casein kinase 1, alpha 1 | 127 |
| 126_CSNK1A1 | casein kinase 1, alpha 1 | 128 |
| 50_CSNK1A1 | casein kinase 1, alpha 1 | 184 |
| 92_CSNK1G3 | casein kinase 1, gamma 3 | 52 |
| 24_CSNK1G3 | casein kinase 1, gamma 3 | 53 |
| | Cell Cycle | |
| 51_mitosis | cell cycle/mitosis | 48 |
| 22_re-entry into mitotic cell cycle | cell cycle/mitosis | 166 |
| 114_CDC2 | cell cycle/mitosis | 169 |
| 114_NEK2 | cell cycle/mitosis | 173 |
| 114_CKS1B | cell cycle | 180 |
| 114_CENPF | cell cycle/mitosis | 181 |
| 114_CENPA | cell cycle/mitosis | 187 |
| 77_Aurora B/RasGAP | cell cycle/mitosis | 234 |
| 100_CDC20 | cell cycle/mitosis | 251 |
| 77_CDCA8 | cell cycle/mitosis | 261 |
| 20_Cyclin D3/CDK11 p58 | cell cycle/G1-S | 446 |
| 100_PRC1 | cell cycle/mitosis | 354 |
| 114_CENPB | cell cycle/mitosis | 359 |
| 100_APC/C/CDC20 | cell cycle/mitosis | 394 |
| 77_Centraspindlin | cell cycle/mitosis | 412 |
| 114_PLK1 | cell cycle/mitosis | 421 |
| 77_cytokinesis | cell cycle/mitosis | 442 |
| 100_CENPE | cell cycle/mitosis | 474 |
| 114_CDC25B | cell cycle/mitosis | 491 |
| 49_PCNA | cell cycle/replication | 363 |
| 30_RBBP7 | cell cycle-Rb binding protein | 379 |
| 40_MNAT1 | component of CAK - cell cycle | 92 |
| 114_CCNB2 | cell cycle/mitosis | 186 |
| 40_CCNH | cyclin H; transcriptional regulation/cell cycle | 19 |
| | DNA damage response | |
| 114_CHEK2 | DNA damage response | 132 |
| 49_RAD50 | DNA damage response | 215 |
| 30_RAD50 | DNA damage response | 216 |
| 49_DNA repair | DNA damage response | 260 |
| 114_BRCA2 | DNA damage response | 388 |
| 49_FA complex/FANCD2/Ubiquitin | DNA damage response | 432 |
| 49_BRCA1/BARD1/RAD51/PCNA | DNA damage response | 449 |
| 40_TFIIH | nucleotide DNA excision repair | 30 |
| 49_FANCE | involved in DSB repair | 22 |
| 49_FANCA | involved in DSB repair | 47 |
| | chromatin remodelling | |
| 114_HIST1H2BA | histone | 347 |
| 112_KAT2B | histone acetyltransferase function | 406 |
| 106_HDAC1 | histone acetyltransferase function | 418 |
| 106_KAT2B | histone acetyltransferase function | 423 |
| 63_KAT2B | histone acetyltransferase function | 425 |
| 47_KAT2B | histone acetyltransferase function | 426 |
| 40_KAT2B | histone acetyltransferase function | 427 |
| 63_I kappa B alpha/HDAC3 | histone deacetylase | 185 |
| 52_HDAC7/HDAC3 | histone deacetylase | 208 |
| 52_HDAC5/ANKRA2 | histone deacetylase | 278 |
| 40_HDAC3 | histone deacetylase | 440 |
| 52_HDAC3 | histone deacetylase | 441 |
| 63_HDAC3 | histone deacetylase | 472 |
| 63_HDAC3/SMRT (N-CoR2) | chromatin remodelling | 370 |
| 63_I kappa B alpha/HDAC1 | chromatin remodelling | 454 |
| | Cell Adhesion | |
| 23_alphaV/beta3 Integrin/Caspase 8 | integrin | 220 |
| 113_ITGAV | integrin | 221 |
| 23_ITGAV | integrin | 222 |
| 2_ITGAV | integrin | 223 |
| 103_ITGAV | integrin | 224 |
| 23_alphaV/beta3 Integrin/Del1 | integrin | 338 |
| 51_ITGB3 | integrin beta 3 | 36 |
| 29_alphaIIb/beta3 Integrin | FN receptor expressed in platelets | 393 |
| 101_alphaIIb/beta3 Integrin | FN receptor expressed in platelets | 395 |
| 84_alphaIIb/beta3 Integrin | FN receptor expressed in platelets | 430 |
| | Proteolysis | |
| 126_PSEN1 | presinilin 1 - protease | 323 |
| 76_PSEN1 | presinilin 1 - protease | 324 |
| 117_PSEN1 | presinilin 1 - protease | 325 |
| | G protein signaling | |
| 16_GDI1 | Rab GDP dissociation inhibitor | 478 |
| 98_RABGGTA | Rab geranylgeranyltransferase | 340 |
| 45_RAP1B | Ras family member | 434 |
| 103_RAP1B | Ras family member | 435 |
| 56_RAP1B | Ras family member | 436 |
| 104_RAP1B | Ras family member | 437 |
| 70_RAP1B | Ras family member | 438 |
| 19_RAP1B | Ras family member | 439 |
| 22_RASA1 | Ras-GAP | 72 |
| 108_RASA1 | Ras-GAP | 73 |
| 19_RASA1 | Ras-GAP | 74 |
| 109_RASA1 | Ras-GAP | 75 |
| 78_RASA1 | Ras-GAP | 76 |
| 43_RASA1 | Ras-GAP | 77 |
| 77_RASA1 | Ras-GAP | 78 |
| 88_RASA1 | Ras-GAP | 79 |
| 7_RASA1 | Ras-GAP | 80 |
| 26_RASA1 | Ras-GAP | 81 |
| 104_RASA1 | Ras-GAP | 82 |
| 91_RASA1 | Ras-GAP | 398 |
| 72_GNG2 | gamma subunit of a trimeric G protein | 51 |
| 58_GNG2 | gamma subunit of a trimeric G protein | 60 |
| 119_GNG2 | gamma subunit of a trimeric G protein | 63 |
| 75_GNG2 | gamma subunit of a trimeric G protein | 64 |
| 24_GNG2 | gamma subunit of a trimeric G protein | 65 |
| 79_GNG2 | gamma subunit of a trimeric G protein | 66 |
| 67_GNG2 | gamma subunit of a trimeric G protein | 67 |
| 52_GNG2 | gamma subunit of a trimeric G protein | 68 |
| 79_GNB1/GNG2 | gamma subunit of a trimeric G protein | 414 |
| 72_GNB1/GNG2 | gamma subunit of a trimeric G protein | 431 |
| 67_G-protein coupled receptor activity | GPCR signaling | 348 |
| 128_mol:GTP | GTP function | 218 |
| 42_mol:GDP | GTP signaling | 336 |
| | RTK/non-RTK signaling | |
| 103_PDGFB-D/PDGFRB | RTK signaling | 112 |
| 83_PDGFB-D/PDGFRB | RTK signaling | 113 |
| 83_PDGFRB | RTK signaling | 114 |
| 103_PDGFRB | RTK signaling | 115 |
| 84_PDGFRB | RTK signaling | 116 |
| 91_PDGFRB | RTK signaling | 134 |
| 82_PDGFB-D/PDGFRB | RTK signaling | 135 |
| 82_PDGFRB | RTK signaling | 136 |
| 104_KIDINS220/CRKL | RTK signaling | 146 |
| 113_CRKL | RTK signaling | 147 |
| 104_CRKL | RTK signaling | 148 |
| 53_CRKL | RTK signaling | 149 |
| 57_CRKL | RTK signaling | 150 |
| 124_CRKL | RTK signaling | 151 |
| 131_CRKL | RTK signaling | 152 |
| 70_CRKL | RTK signaling | 153 |
| 91_Bovine Papilomavirus E5/PDGFRB | RTK signaling | 161 |
| 46_GRB10 | RTK signaling | 380 |
| 7_GRB10 | RTK signaling | 381 |
| 88_GRB10 | RTK signaling | 382 |
| 91_GRB10 | RTK signaling | 383 |
| 88_GRB14 | RTK signaling | 404 |
| 108_GRB14 | RTK signaling | 405 |
| 2_GRB10 | RTK signaling | 471 |
| 135_EGFR | RTK signaling | 479 |
| 48_EGFR | RTK signaling | 480 |
| 38_EGFR | RTK signaling | 481 |
| 71_EGFR | RTK signaling | 482 |
| 58_EGFR | RTK signaling | 483 |
| 17_EGFR | RTK signaling | 484 |
| 76_EGFR | RTK signaling | 485 |
| 29_EGER | RTK signaling | 486 |
| 72_EGFR | RTK signaling | 497 |
| 84_EGFR | RTK signaling | 499 |
| 84_FER | tyrosine kinase | 217 |
| 46_PTK2 | FAK homologue - cell motility | 156 |
| 109_PTK2 | FAK homologue - cell motility | 157 |
| 72_PTK2 | FAK homologue - cell motility | 397 |
| 119_PTK2 | FAK homologue - cell motility | 411 |
| 7_FRS2 | fibroblast growth factor substrate | 461 |
| 2_FRS2 | fibroblast growth factor substrate | 462 |
| 104_FRS2 | fibroblast growth factor substrate | 463 |
| 87_ERBB2IP | negatively regulates ErbB2 | 228 |
| | PI3K/AKT signaling | |
| 51_AKT1 | signaling; tumor cell survival | 91 |
| 44_AKT1 | signaling; tumor cell survival | 143 |
| 108_PIK3R1 | signaling; tumor cell survival | 269 |
| 72_PIK3R1 | signaling; tumor cell survival | 274 |
| 94_PIK3R1 | signaling; tumor cell survival | 275 |
| 122_PIK3R1 | signaling; tumor cell survival | 276 |
| 22_PIK3R1 | signaling; tumor cell survival | 277 |
| 45_PIK3R1 | signaling; tumor cell survival | 279 |
| 103_PIK3R1 | signaling; tumor cell survival | 281 |
| 2_PIK3R1 | signaling; tumor cell survival | 282 |
| 23_PIK3R1 | signaling; tumor cell survival | 283 |
| 88_PIK3R1 | signaling; tumor cell survival | 284 |
| 101_PIK3R1 | signaling; tumor cell survival | 285 |
| 104_PIK3R1 | signaling; tumor cell survival | 286 |
| 79_PIK3R1 | signaling; tumor cell survival | 287 |
| 51_PIK3R1 | signaling; tumor cell survival | 288 |
| 109_PIK3R1 | signaling; tumor cell survival | 289 |
| 117_PIK3R1 | signaling; tumor cell survival | 290 |
| 124_PIK3R1 | signaling; tumor cell survival | 291 |
| 7_PIK3R1 | signaling; tumor cell survival | 292 |
| 113_PIK3R1 | signaling; tumor cell survival | 293 |
| 69_PIK3R1 | signaling; tumor cell survival | 294 |
| 116_PIK3R1 | signaling; tumor cell survival | 295 |
| 119_PIK3R1 | signaling; tumor cell survival | 296 |
| 131_PIK3R1 | signaling; tumor cell survival | 297 |
| 80_PIK3R1 | signaling; tumor cell survival | 298 |
| 91_PIK3R1 | signaling; tumor cell survival | 299 |
| 135_PIK3R1 | signaling; tumor cell survival | 300 |
| 68_PIK3R1 | signaling; tumor cell survival | 301 |
| 84_PIK3R1 | signaling; tumor cell survival | 302 |
| 46_PIK3R1 | signaling; tumor cell survival | 303 |
| 3_PIK3R1 | signaling; tumor cell survival | 304 |
| 57_PIK3R1 | signaling; tumor cell survival | 305 |
| 19_PIK3R1 | signaling; tumor cell survival | 306 |
| 43_PIK3R1 | signaling; tumor cell survival | 307 |
| 70_PIK3R1 | signaling; tumor cell survival | 311 |
| 38_PIK3R1 | signaling; tumor cell survival | 320 |
| 93_PIK3R1 | signaling; tumor cell survival | 321 |
| 55_PIK3R1 | signaling; tumor cell survival | 339 |
| 74_PIK3R1 | signaling; tumor cell survival | 444 |
| 9_PIK3R1 | signaling; tumor cell survival | 460 |
| 51_RPS6KB1 | ribosomal protein S6 kinase - signaling | 50 |
| 16_RPS6KA4 | ribosomal protein S6 kinase - signaling | 378 |
| 51_FRAP1 | AKA:mTOR - signaling | 98 |
| 51_mol:PI-3-4-5-P3 | pro-survival | 97 |
| 51_PI3K | pro-survival | 138 |
| | TGFb signaling | |
| 105_SMAD5 | TGFb signaling | 174 |
| 105_SMAD5/SMAD5/SMAD4 | TGFb signaling | 197 |
| 105_SMAD6/SMURF1/SMAD5 | TGFb signaling | 214 |
| 105_BMP4 | TGFb signaling | 229 |
| 105_SMAD9 | TGFb signaling | 310 |
| 105_SMAD5/SKI | TGFb signaling | 322 |
| 105_SMAD8A/SMAD8A/SMAD4 | TGFb signaling | 346 |
| 105_CHRDL1 | BMP4 antagonist | 498 |
| | ser/thr phosphatase | |
| 131_mol:PP2 | ser/thr phosphatase | 312 |
| 43_PPAP2A | ser/thr phosphatase | 500 |
| 120_PPP2R5D | PP2A - ser/thr phosphatase | 40 |
| 77_PPP2R5D | PP2A - ser/thr phosphatase | 41 |
| 26_PPP2R5D | PP2A - ser/thr phosphatase | 42 |
| 100_PPP2CA | PP2A - ser/thr phosphatase | 122 |
| 105_PPM1A | PP2C family member - ser/thr phosphatase | 272 |
| 115_PPM1A | PP2C family member - ser/thr phosphatase | 273 |
| | Transcription Factor | |
| 106_positive regulation of transcription | transcription | 256 |
| 30_MAX | transcription factor | 39 |
| 63_MAX | transcription factor | 46 |
| 112_MAX | transcription factor | 119 |
| 95_NFAT1/CK1 alpha | transcription factor | 191 |
| 114_ETV5 | transcription factor | 211 |
| 95_NFAT4/CK1 alpha | transcription factor | 241 |
| 63_GATA2 | transcription factor | 257 |
| 106_GATA2 | transcription factor | 258 |
| 52_GATA2 | transcription factor | 259 |
| 112_FOXG1 | transcription factor | 262 |
| 112_GSC | transcription factor | 328 |
| 63_GATA2/HDAC3 | transcription factor | 337 |
| 52_MEF2C | transcription factor | 341 |
| 14_FOXA1 | transcription factor | 349 |
| 112_MYC | transcription factor | 357 |
| 30_MYC | transcription factor | 362 |
| 63_GATA1/HDAC3 | transcription factor | 368 |
| 52_GATA2/HDAC5 | transcription factor | 371 |
| 105_ENDOFIN/SMAD1 | transcription factor | 372 |
| 52_GATA1 | transcription factor | 377 |
| 106_EGR1 | transcription factor | 453 |
| 16_USF1 | transcription factor | 468 |
| 114_MYC | transcription factor | 470 |
| 114_FOXM1 | transcription factor | 490 |
| 39_FOS | transcription factor - mitogenic signaling | 212 |
| 37_FOS | transcription factor - mitogenic signaling | 227 |
| 30_FOS | transcription factor - mitogenic signaling | 237 |
| 72_FOS | transcription factor - mitogenic signaling | 242 |
| 43_FOS | transcription factor - mitogenic signaling | 246 |
| 126_FOS | transcription factor - mitogenic signaling | 247 |
| 109_FOS | transcription factor - mitogenic signaling | 248 |
| 93_FOS | transcription factor - mitogenic signaling | 249 |
| 70_CAMK2A | transcription factor - mitogenic signaling | 250 |
| 87_FOS | transcription factor - mitogenic signaling | 267 |
| 110_FOS | transcription factor - mitogenic signaling | 407 |
| 10_FOS | transcription factor - mitogenic signaling | 419 |
| 112_FOS | transcription factor - mitogenic signaling | 476 |
| 22_AP-1 | transcription factor; mitogenic response | 154 |
| 51_EGR2 | transcription factor; regulates ErbB2 exspression | 45 |
| 40_CDK7 | transcription initiation; DNA repair | 29 |
| | ubiquitination | |
| 41_beta TrCP1/SCF ubiquitin ligase | ubiquitination | 56 |
| complex | | |
| 41_FBXW11 | ubiquitination | 57 |
| 69_beta TrCP1/SCF ubiquitin ligase | ubiquitination | 102 |
| complex | | |
| 63_beta TrCP1/SCF ubiquitin ligase | ubiquitination | 103 |
| complex | | |
| 35_beta TrCP1/SCF ubiquitin ligase | ubiquitination | 104 |
| complex | | |
| 126_FBXW11 | ubiquitination | 105 |
| 63_FBXW11 | ubiquitination | 106 |
| 50_FBXW11 | ubiquitination | 107 |
| 100_FBXW11 | ubiquitination | 108 |
| 35_FBXW11 | ubiquitination | 109 |
| 69_FBXW11 | ubiquitination | 110 |
| 106_proteasomal ubiquitin-dependent | ubiquitination | 177 |
| protein catabolic process | | |
| 41_proteasomal ubiquitin-dependent | ubiquitination | 355 |
| protein catabolic process | | |
| 63_proteasomal ubiquitin-dependent | ubiquitination | 448 |
| protein catabolic process | | |
| 51_CBL | adaptor protein; regulates ubiquitination of RTKs | 183 |
| | Wnt signaling | |
| 38_CTNNA1 | Wnt signaling | 263 |
| 45_CTNNA1 | Wnt signaling | 264 |
| 103_CTNNA1 | Wnt signaling | 265 |
| 71_CTNNA1 | Wnt signaling | 266 |
| 75_FZD6 | Wnt signaling | 360 |
| 111_FZD6 | Wnt signaling | 361 |
| 126_DKK1/LRP6/Kremen 2 | Wnt signaling | 389 |
| 50_DKK1/LRP6/Kremen 2 | Wnt signaling | 390 |
| 126_Axin1/APC/beta catenin | Wnt signaling | 392 |
| 126_WNT1 | Wnt signaling | 464 |
| 50_WNT1 | Wnt signaling | 466 |
| | Other | |
| 51_AICDA | activation-induced cytidine deaminase | 2 |
| 44_ABCB1 | ABC transporter - multidrug resistance | 428 |
| 131_LRP8 | apolipoprotein E receptor | 332 |
| 120_LRP8 | apolipoprotein E receptor | 333 |
| 51_ALOX15 | arachidonate 15-lipoxygenase | 5 |
| 14_TTR | carrier protein | 495 |
| 87_CHRNA1 | cholinergic receptor | 455 |
| 33_LNPEP | cleaves peptide hormones | 416 |
| 88_F2RL2 | coagulation factor | 245 |
| 51_COL1A1 | collagen 1A1; ECM | 192 |
| 51_COL1A2 | collagen 1A2; ECM | 209 |
| 95_NUP214 | component of the nuclear pore complex | 327 |
| 105_NUP214 | component of the nuclear pore complex | 329 |
| 115_NUP214 | component of the nuclear pore complex | 330 |
| 40_positive regulation of DNA binding | DNA binding?? | 124 |
| 77_Chromosomal passenger complex | DNA function | 352 |
| 77_Chromosomal passenger | DNA function | 410 |
| complex/EVI5 | | |
| 30_BLM | DNA helicase | 350 |
| 24_RAB23 | endocytosis; vesicular transport | 196 |
| 48_EDN1 | endothelin 1 - vasoconstriction | 364 |
| 10_GADD45B | growth arrest and DNA damage inducible gene | 422 |
| 89_GUCA1B | guanylate cyclase | 429 |
| 114_HSPA1B | heat shock protein | 54 |
| 47_mol:Lysophosphatidic acid | LPA signaling | 465 |
| 87_myelination | mucscle function | 353 |
| 105_RGMB | neuronal function | 255 |
| 7_GFRA1 | neurotrophic factor | 374 |
| 51_OPRM1 | opioid receptor | 14 |
| 62_negative regulation of phagocytosis | phagocytosis | 244 |
| 23_PI4KA | phosphatidylinositol 4-kinase | 163 |
| 89_PDE6A/B | phosphodiesterase | 433 |
| 89_PDE6A | phosphodiesterase | 469 |
| 43_GO:0007205 | PKC signaling | 387 |
| 95_PRKCH | PKC-eta (epithelial specifc) | 253 |
| 45_KLHL20 | pleoitrophic | 384 |
| 58_PTGDR | prostaglandin D2 receptor | 239 |
| 58_PGD2/DP | prostaglandin D2 synthase | 326 |
| 105_ZFYVE16 | protein trafficking | 69 |
| 33_VAMP2 | protein trafficking | 238 |
| 21_VAMP2 | protein trafficking | 308 |
| 102_EXOC5 | protein trafficking | 309 |
| 71_CYFIP2 | putative role in adhesion/apoptosis | 94 |
| 45_CYFIP2 | putative role in adhesion/apoptosis | 95 |
| 52_ANKRA2 | putative role in endocytosis | 49 |
| 108_mol:ROS | reactive oxygen species | 167 |
| 31_oxygen homeostasis | redox | 268 |
| 54_NPHS1 | renal function | 496 |
| 51_RETNLB | resistin like beta | 4 |
| 51_TFF3 | secreted protein normally found in the GI mucosa | 21 |
| 52_SRF | serum response factor; immediate early gene | 141 |
| 51_SOCS1 | Stat signaling | 139 |
| 51_SOCS3 | Stat signaling | 376 |
| 106_SENP1 | sumoylation | 494 |
| 16_EIF4EBP1 | translation | 366 |

While all of the above pathway entities, when differentially expressed relative to normal (overexpressed or underexpressed) may serve as indicators for an immune suppressed tumor, it is contemplated that only a fraction may be analyzed. For example, suitable tests may analyze at least 10%, or at least 20%, or at least 30%, or at least 40%, or at least 50%, or at least 60%, or at least 70%, or at least 80%, or at least 90% of the genes/pathway entities listed in Tables 1-4. Alternatively, contemplated tests may also use specific genes of the genes/pathway entities listed in Tables 1-4, and especially one or more of pathway elements selected form the group consisting of IL12B, IFNG, PSMA3, THY1, CCL17, PRKCQ, NFATC3, NFATC2, CCL11, CCL26, IFNAR2, SQSTM1, IRAK4, NFKBIA, IL6ST, MAP3K1, IRF1, IRF9, PTGS2, IL4, IL5, IGHG3, IL4R, IL13RA2, PIGR, IL13RA1, STAT6, FCER2, IGHG1, IL10, STAT5A, PRKCE, CSF1R, ARG1, LTA, SELP, FKBP3, LCP2, and DOK2. For example, such list may include at least two, at least three, at least four, at least five, at least ten, at least 15, or at least 20 of IL12B, IFNG, PSMA3, THY1, CCL17, PRKCQ, NFATC3, NFATC2, CCL11, CCL26, IFNAR2, SQSTM1, IRAK4, NFKBIA, IL6ST, MAP3K1, IRF1, IRF9, PTGS2, IL4, IL5, IGHG3, IL4R, IL13RA2, PIGR, IL13RA1, STAT6, FCER2, IGHG1, IL10, STAT5A, PRKCE, CSF1R, ARG1, LTA, SELP, FKBP3, LCP2, and DOK2.

In addition, contemplated assays need not only be limited to single pathway elements, but may also include complexes of pathway elements, and especially one or more complexes selected from the group consisting of IFN-gamma/IRF1, STAT6 (dimer)/PARP14, IL4/IL4R/JAK1, IL4R/JAK1, STAT6 (dimer)/ETS1, PI3K/BCAP/CD19, IL4/IL4R/JAK1/IL2Rgamma/JAK3/DOK2, IL4/IL4R/JAK1/IL2Rgamma/JAK3/SHIP, IL4/IL4R/JAK1/IL13RA1/JAK2, IL4/IL4R/JAK1/IL2Rgamma/JAK3/SHC/SHIP, IL4/IL4R/JAK1/IL2Rgamma/JAK3/FES/IRS2, IL4/IL4R/JAK1/IL2Rgamma/JAK3, IL4/IL4R/JAK1/IL2Rgamma/JAK3/SHC/SHIP/GRB2, IL4/IL4R/JAK1/IL2Rgamma/JAK3/IRS1, IL4/IL4R/JAK1/IL2Rgamma/JAK3/FES, IL4/IL4R/JAK1/IL2Rgamma/JAK3/SHP1 (or any combination of at least two, at least three, at least four, at least five, or at least ten complexes).

In addition, the differentially expressed genes may include highly expressed genes, and especially FOXM1. Still further contemplated differentially expressed genes include non-immune genes that encode a protein involved in at least one of mitogenic signaling, stress signaling, apoptosis, calcium/calmodulin signaling, G-protein signaling, PI3K/AKT signaling, RTK signaling, Wnt signaling, and cAMP signaling, or non-immune genes encoding a protein that is involved in at least one of cell cycle control, DNA damage response, and chromatin remodeling as shown in Tables 2 and 4 above. For example, suitable contemplated non-immune genes include at least one, at least two, at least three, at least four, at least five, at least ten MAPK1, MAPK14, NRP2, HIF1A, CALM1, CREB1, CSNK1A1, CSNK1G3, CCNH, FANCE, FANCA, TFIIH, ITGB3, RASA1, GNG2, PDGFRB, AKT1, and PIK3R1.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of predicting a likely therapeutic outcome for immune therapy of a cancer with a checkpoint inhibitor, comprising:
obtaining omics data from a tumor of the patient, wherein the omics data comprise at least one of whole genome sequencing data and RNA sequencing data;
using pathway analysis to identify from the omics data a plurality of highly expressed genes in a plurality of immune related pathways having a plurality of respective pathway elements;
associating the highly expressed genes with likely response of the cancer to treatment with the checkpoint inhibitor when the highly expressed genes are indicative of a Th2/humoral response and a low Th1/Th2 ratio; and
updating or generating a patient record with an indication of the likely response of the cancer to treatment with the checkpoint inhibitor when the highly expressed genes are indicative of a Th2/humoral response and a low Th1/Th2 ratio.

2. The method of claim 1 wherein the immune related pathways are selected from the group consisting of an immune cell function pathway, a pro-inflammatory signaling pathway, and an immune suppression pathway.

3. The method of claim 1 wherein the pathway element control activity of at least one of Th1 differentiation, Th2 differentiation, B cell differentiation, macrophage differentiation, T cell activation, and an immunoproteasome.

4. The method of claim 1 wherein the pathway element control activity of at least one of NFkB, an IFNalpha responsive gene.

5. The method of claim 1 wherein the pathway element is a cytokine.

6. The method of claim 1 wherein the cytokine is selected form the group consisting of IL12 beta, IFNgamma, IL4, IL5, and IL10.

7. The method of claim 1 wherein the pathway element is a chemokine.

8. The method of claim 1 wherein the chemokine is selected from the group consisting of CCL17, CCL11, and CCL26.

9. The method of claim 1 wherein the pathway element is selected form the group consisting of IL12B, IFNG, PSMA3, THY1, CCL17, PRKCQ, NFATC3, NFATC2, CCL11, CCL26, IFNAR2, SQSTM1, IRAK4, NFKBIA, IL6ST, MAP3K1, IRF1, IRF9, PTGS2, IL4, IL5, IGHG3, IL4R, IL13RA2, PIGR, IL13RA1, STAT6, FCER2, IGHG1, IL10, STAT5A, PRKCE, CSF1R, ARG1, LTA, SELP, FKBP3, LCP2, and DOK2.

10. The method of claim 1 wherein the pathway element is a complex selected form the group consisting of IFN-gamma/IRF1, STAT6 (dimer)/PARP14, IL4/IL4R/JAK1, IL4R/JAK1, STAT6 (dimer)/ETS1, PI3K/BCAP/CD19, IL4/IL4R/JAK1/IL2Rgamma/JAK3/DOK2, IL4/IL4R/JAK1/IL2Rgamma/JAK3/SHIP, IL4/IL4R/JAK1/IL13RA1/JAK2, IL4/IL4R/JAK1/IL2Rgamma/JAK3/SHC/SHIP, IL4/IL4R/JAK1/IL2Rgamma/JAK3/FES/IRS2, IL4/IL4R/JAK1/IL2Rgamma/JAK3, IL4/IL4R/JAK1/IL2Rgamma/JAK3/SHC/SHIP/GRB2, IL4/IL4R/JAK1/IL2Rgamma/JAK3/IRS1, IL4/IL4R/JAK1/IL2Rgamma/JAK3/FES, IL4/IL4R/JAK1/IL2Rgamma/JAK3/SHP1.

11. The method of claim 1 wherein the omics data further comprise at least one of siRNA data, DNA methylation status data, transcription level data, and proteomics data.

12. The method of claim 1 wherein the pathway analysis comprises PARADIGM analysis.

13. The method of claim 1 wherein the omics data are normalized against the same patient.

14. The method of claim 1 wherein the checkpoint inhibitor is a CTLA-4 inhibitor or a PD-1 inhibitor.

15. The method of claim 1 wherein the cancer is a breast cancer, and wherein the highly expressed genes further include FOXM1.

16. The method of claim 1 wherein the highly expressed genes further include non-immune genes encoding a protein involved in at least one of mitogenic signaling, stress signaling, apoptosis, calcium/calmodulin signaling, G-protein signaling, PI3K/AKT signaling, RTK signaling, Wnt signaling, and cAMP signaling.

17. The method of claim 1 wherein the highly expressed genes further include non-immune genes encoding a protein involved in at least one of cell cycle control, DNA damage response, and chromatin remodeling.

18. The method of claim 1 wherein the highly expressed genes further include non-immune genes selected from the group consisting of MAPK1, MAPK14, NRP2, HIF1A, CALM1, CREB1, CSNK1A1, CSNK1G3, CCNH, FANCE, FANCA, TFIIH, ITGB3, RASA1, GNG2, PDGFRB, AKT1, and PIK3R1.

19. The method of claim 1 wherein the likely therapeutic outcome is predicted prior to therapy with the checkpoint inhibitor.

20. The method of claim 1 wherein the immune therapy further comprises administration of at least one of a genetically modified virus and a genetically modified NK cell.
